(19) United States
(12) Patent Application Publication
(10) Pub. No.: US 2026/0085266 A1
(43) Pub. Date: Mar. 26, 2026

(54) METHOD FOR PERFORMING A BIOPROCESS ON LIQUID IMMUNE OR NAIVE CELL CULTURES TO OBTAIN PROCESSED CELL CULTURES

(71) Applicant: The Automation Partnership (Cambridge) Ltd, Royston, Hertfordshire (GB)

(72) Inventors: Adrian Stacey, Cambridge (GB); Fanny Prouté, Cambridge (GB)

(21) Appl. No.: 19/114,269

(22) PCT Filed: Dec. 13, 2022
(86) PCT No.: PCT/EP2022/085651

§ 371 Date: Mar. 21, 2025

(30) Foreign Application Priority Data
Sep. 21, 2022 (WO) PCT/IB2022/000491

(51) Int. Cl. C12M 3/00; C12M 1/12; C12M 1/36; C12N 5/078; C12N 5/10; C12N 15/86; C12N 15/87

(52) U.S. Cl. CPC C12M 23/42; C12M 23/44; C12M 37/04; C12M 41/48; C12N 5/0634; C12N 5/10; C12N 15/86; C12N 15/87; C12N 2510/00

(57) ABSTRACT

Method for performing a bioprocess to obtain processed cell cultures, wherein the processed cell cultures are destined for autologous or allogenic cell therapy, wherein the bioprocess is performed on an integrated bioprocessing system, wherein the bioprocess comprises a sequence of processing steps, wherein the processing steps each comprise at least one operation, wherein the bioprocess system comprises a base structure and preconfigurable cartridges, wherein the bioprocess system performs operations of the bioprocess via an interaction of the base structure with the cartridges, wherein the cartridges and the base structure comprise matching standardized interfaces for an interaction of the base structure with the respective cartridge, wherein the bioprocess system performs at least two operations of the bioprocess inside at least two differently preconfigured cartridges by an interaction of the base structure with the cartridges via the same base structure interface and/or identical base structure interfaces and matching cartridge interfaces.

### CROSS-REFERENCE TO RELATED APPLICATIONS

This application is a national stage application under 35 U.S.C. 371 of International Patent Application Serial No. PCT/EP2022/085651, entitled “Method for Performing a Bioprocess on Liquid Immune or Naïve Cell cultures to Obtain Processed Cell Cultures,” filed Dec. 13, 2022, which claims priority from International Patent Application Serial No. PCT/IB2022/000491, entitled “Method for Performing a Bioprocess on Liquid Immune Cell Cultures Comprising a Sequence of Processing Steps,” filed Sep. 21, 2022, the disclosures of which is incorporated herein by reference.

### FIELD OF THE TECHNOLOGY

Various embodiments relate to a method for performing a bioprocess on liquid immune or naive cell cultures to obtain processed cell cultures, to a cartridge, to a base structure and to a bioprocessing system.

### SUMMARY

The term “bioprocess” presently represents a biotechnological process, in particular a biotechnological process involving the use of immune cell cultures or naive cell cultures, including stem cell cultures. One or more processing steps might be performed on each cell culture. Hence, a bioprocess in this sense might refer to a manufacturing process that involves a sequence of processing steps performed on a cell culture which ultimately will lead to a final product.

The proposed method may be used in the area of cell and gene therapy, including allogenic or autologous production of genetically modified immune cells. Exemplarily, the method may be applied to manufacture autologous T cells that are modified to express a chimeric antigen receptor (CAR). These cells might be used for the treatment of various types of hematologic malignancies, including different types of leukemia (blood cancer). Other cell therapies based on naive cells, in particular stem cells and their derivates are also of interest. For biotechnological processes involving the use of liquid immune or naive cell cultures, process flexibility is especially relevant. The starting material might be quite heterogeneous regarding its composition, for example because each patient is in a different condition (e.g., in terms of disease progression or in terms of genetic make-up and history of their immune system). The starting material might also be heterogeneous regarding its composition because of cell cultures of different donors being combined into one initial starting material. Hence, depending on the source of the liquid cell culture, various parameters, including for example the type and concentration of different cell types, the overall cell viability and vitality and/or the amount and type of impurities within the liquid cell culture might vary. Additionally, depending on the bioprocess to be performed, the cell culture might comprise different types of immune cells in different amounts (e.g., T cells, dendritic cells or immune cells in different developmental stages including naïve T cells). Partly because of the reasons mentioned above, the sequence of processing steps performed on each liquid immune cell culture will need to be adjusted to the individual features of the cell culture. In addition, certain steps of the bioprocess might need to be flexibly adapted and tailored to each individual cell culture. For example, the type of genetic modification of the cell culture might be different as patients may respond differently to a certain genetic modification. Accordingly, the bioprocess will differ in the way genetic modification is performed. Also, exemplarily, a bioprocess involving the genetic modification of an immune cell culture will require a sequence of processing steps that is different from the sequence of processing steps required in a bioprocess not involving the genetic modification of an immune cell culture. Besides steps being flexibly adapted, steps may also be omitted completely. This requires that a method for performing a bioprocess on liquid immune cell cultures can be adapted in a flexible manner.

While process flexibility is very important, cost-efficiency is also an important aspect during processing of liquid immune or naive cell cultures. A reason for this is that the requirements from regulatory authorities are very demanding. For example, operators involved in the process will need to be extensively trained, especially when performing manual processes. In addition, sterility must be maintained during the manufacturing process, because the processed cells need to be viable and must not include contaminants when being administered to the recipient. Partly for the reasons mentioned, the overall costs for each bioprocess performed are very high. Additionally, in approaches, where one single device is used to sequentially perform all processing steps on an initial cell culture, the facility footprint is usually large, as one device is required to process one cell culture at a time. Further, the throughput is lowered, as only one liquid cell culture is processed within one device. Also, as the sequence of processing steps is typically predefined, for example by the tubing set installed, adaptation of such devices to changes in the sequence or type of processing steps performed is rather demanding. Nevertheless, these end-to-end systems provide a system wherein all media are kept within a closed inner volume, which supports maintenance of sterility.

Cost-efficiency is particularly related to the operating costs; hence, it is desirable to enable the processing of more than one cell culture in parallel. This also increases the throughput, which is important given the fact that processing of a single initial cell culture might take several days or even weeks. However, when processing more than one cell culture in parallel, it needs to be ensured that no cross-contamination of different cell cultures occurs.

Another important aspect is redundancy, which also relates to process robustness and process reliability. As mentioned above and especially when applied for cancer treatment, processing of a cell culture might take considerable time with patients typically requiring treatment urgently. Hence, it needs to be ensured that the process does not fail. Process failure might result in the process having to be repeated, which would require further time and/or additional starting material needed. However, especially in autologous approaches, it might not be possible to obtain any further starting material from the patient.

WO 2021/212124 A1, that builds the basis of some embodiments, is related to a method. This reference discloses a method for the modular and parallelized processing of liquid immune cell cultures on an integrated bioprocessing system. The method comprises a sequence of processing steps, which are being performed within specific unit operation stations of the bioprocessing system. Although the bioprocessing system shows a certain degree of standardization in the form of a rack receiving all components necessary to perform the unit operations, the unit operation stations as such are each highly customized with regard to the unit operation to be performed.

Accordingly, the configuration of each complete unit operation station is dependent on the type of unit operation performed at the respective unit operation station. Consequently, also the infrastructure (e.g., reservoirs for raw materials and/or waste materials) provided at a unit operation station depends on the unit operation that is being performed at the respective unit operation station. Additionally, the interaction between a transport system and each unit operation station depends on the type of unit operation performed at the respective unit operation station. With the resulting, high degree of customization regarding the unit operation stations, the flexibility regarding the definition of the process steps is high; however, the potential for increasing efficiency by automation stays comparably low.

Various embodiments are based on the problem of improving the known method such that the potential for automation is increased without compromising the flexibility with respect to the definition of process steps.

The above-noted object is solved by various features described herein.

A main realization of some embodiments is that by providing a standardized structure based on individually preconfigured cartridges, a number of different processing steps can be performed by an integrated bioprocessing system in a flexible manner while maintaining sterility. The efficiency and flexibility of the overall bioprocess can thereby be increased. The main idea is to separate those parts of the system that can be reused from the liquids and the liquid containing single-use components and providing a modular bioprocessing system that allows combining multi-use and single-use components as needed.

Proposed is a method for performing a bioprocess on liquid immune or naive cell cultures to obtain processed cell cultures, wherein the processed cell cultures are destined for autologous or allogenic cell therapy, wherein the bioprocess is performed on an integrated bioprocessing system, wherein the bioprocess comprises a sequence of processing steps, wherein the processing steps each comprise at least one operation, wherein the bioprocess system comprises a base structure and preconfigurable cartridges, wherein the bioprocess system performs operations of the bioprocess via an interaction of the base structure with the cartridges, wherein the cartridges and the base structure comprise matching standardized interfaces for an interaction of the base structure with the respective cartridge, wherein the bioprocess system performs at least two operations of the bioprocess inside at least two differently preconfigured cartridges by an interaction of the base structure with the cartridges via the same base structure interface and/or identical base structure interfaces and matching cartridge interfaces.

The term “different” is to be understood as relating to a functionally relevant difference and for example not to purely optical deviations. The differently preconfigured cartridges may have a different number of tubes, substantially different tube lengths, different functional devices, a different number of inlets or outlets, different containers for receiving and/or providing a liquid or the like.

The term “standardized” means that differently configured cartridges for different operations can be used via the same base structure interface. In various embodiments, the cartridge interfaces are identical.

The term “preconfigured” means that the respective cartridge is configured outside the bioprocessing system, for example manually or by a manufacturer of the cartridge or that the cartridge is configured by the bioprocessing system automatically by adding components. Configuration is not to be understood as only relating to a digital configuration or minimal changes or in particular changes during use of the cartridge. The preconfiguration mechanically changes the state of the cartridge from a non-usable state to a usable state. In particular, the preconfiguration comprises adding, in particular changing (removing and adding), a cartridge fluidic structure.

Various embodiments relate combinations of bioprocesses, bioprocessing steps and operations performed by the bioprocessing system.

Various embodiments of the cartridges are provided herein.

Various embodiments of the interfaces are provided herein.

Various embodiments describe receptacles used by the bioprocessing system.

Various embodiments relate to different cartridges adapted for a number of processing steps.

Various embodiments relate to miscellaneous details of the method.

Various embodiments relate to a cartridge for use in the proposed method.

All explanations given with regard to the proposed method are fully applicable.

Another teaching according to various embodiments relate to a base structure for use in the proposed method.

All explanations given with regard to the proposed method are fully applicable.

Another teaching according to various embodiments relate to a bioprocessing system for use in the proposed method.

All explanations given with regard to the proposed method are fully applicable.

Various embodiments provide a method for performing a bioprocess on liquid immune or naive cell cultures to obtain processed cell cultures, wherein the processed cell cultures are destined for autologous or allogenic cell therapy, wherein the bioprocess is performed on an integrated bioprocessing system, wherein the bioprocess comprises a sequence of processing steps, wherein the processing steps each comprise at least one operation, wherein the bioprocess system comprises a base structure and preconfigurable cartridges, wherein the bioprocess system performs operations of the bioprocess via an interaction of the base structure with the cartridges, wherein the cartridges and the base structure comprise matching standardized interfaces for an interaction of the base structure with the respective cartridge, wherein the bioprocess system performs at least two operations of the bioprocess inside at least two differently preconfigured cartridges by an interaction of the base structure with the cartridges via the same base structure interface and/or identical base structure interfaces and matching cartridge interfaces.

In various embodiments, the two operations of the bioprocess are operations of different processing steps, or, that the bioprocessing system performs at least two operations that are part of different bioprocesses in parallel in identically preconfigured cartridges, or, that the bioprocessing system performs at least two operations that are part of different bioprocesses in parallel in differently preconfigured cartridges.

In various embodiments, the bioprocessing system performs at least two bioprocesses or at least three bioprocesses or at least five bioprocesses or at least ten bioprocesses, each comprising at least two or at least three or at least four different processing steps in parallel, wherein the bioprocessing system performs at least one operation per processing steps, wherein the bioprocessing system performs those operations in at least two or at least three or at least four differently preconfigured cartridges per bioprocess.

In various embodiments, the operations comprise operations of one or more of the processing steps “enrichment”, “selection”, “activation”, “loading”, “genetic modification”, “expansion”, “formulation”, “fill”, “wash” and “separation”, or,
that the processing steps comprise one or more of the processing steps “enrichment”, “selection”, “activation”, “loading”, “genetic modification”, “expansion”, “formulation”, “fill”, “wash” and “separation”.

In various embodiments, the bioprocessing system comprises single step cartridges, whereby the bioprocessing system performs only operations of a single processing step inside the single step cartridges, or, that the bioprocessing system comprises shared cartridges, whereby the bioprocessing system performs operations of, in particular consecutive, processing steps inside the shared cartridges.

In various embodiments, the preconfigured cartridges comprise a preconfigured cartridge fluidic structure, or, that the preconfigured cartridges comprise a, in particular preconfigured, functional device for the operation performed inside the cartridge.

In various embodiments, the standardized base structure interface comprises an active, energy transmitting interface, that the base structure via the active base structure interface and the matching cartridge interface transfers one or more of mechanical, pneumatic and electrical energy to the cartridge.

In various embodiments, the bioprocessing system comprises receptacles, that the receptacles are preconfigured with receptacle fluidic structures for containing and providing or receiving the cell cultures or consumables that the bioprocessing system uses in at least some of the operations, and, that the receptacles comprise fluid interfaces identical to the fluid interfaces of the cartridges.

In various embodiments, the bioprocessing system performs one or more or all operations of the processing step “enrichment”, the processing step “wash” and the processing step “separation” via the base structure inside a preconfigured filter cartridge with at least one filter as a functional device inside the cartridge.

In various embodiments, the bioprocessing system performs one or more or all operations of the processing step “activation” via the base structure inside a preconfigured activation cartridge with at least one pump as a functional device inside or outside the cartridge.

In various embodiments, the bioprocessing system performs one or more or all operations of the processing step “genetic modification” via the base structure inside a preconfigured genetic modification cartridge, and, that the activation cartridge and the genetic modification cartridge are one shared cartridge.

In various embodiments, the bioprocessing system performs one or more or all operations of the processing step “genetic modification” via the base structure inside the filter cartridge as a shared cartridge.

In various embodiments, the bioprocessing system performs the genetic modification by viral transduction or electroporation or nanoparticle-based delivery.

In various embodiments, the cartridges or the receptacles are moved by the bioprocessing system automatically.

In various embodiments, an electronic process control of the bioprocessing system may repeat a processing step or an operation if a criterion for the cell culture is not met during or after the processing step, thereby flexibly reconfiguring the bioprocess, or, that variations of processing steps may be performed via the same standardized base structure interface by differently preconfiguring the cartridge without reconfiguring the base structure.

In various embodiments, the standardized interfaces are immovable.

In various embodiments, the cartridges have no energy source or only a battery that does not drive one of the functional devices, or, that the cartridges are partially or fully made out of plastic or 3D-printed or do not comprise any fluidic structure prior to being preconfigured.

Various embodiments provide a cartridge for use in the method as described herein.

In various embodiments, the cartridge is preconfigured with a sterilized and sealed fluidic structure.

Various embodiments provide a bioprocessing system for use in the method as described herein.

### BRIEF DESCRIPTION OF THE DRAWINGS

In the following, embodiments are explained with respect to the drawing. The drawing shows in

FIG. 1, a schematic representation of a proposed integrated bioprocessing system,

FIG. 2, in a perspective view a) a cartridge as such without the cartridge fluidic structure and b) in an explosive representation a cartridge with a cartridge fluidic structure and a unit operation station,

FIG. 3, a unit operation station of the bioprocessing system in FIG. 1 during performing a unit operation,

FIG. 4, the bioprocessing system of FIG. 1 a) in a sectional view along line IV-IV and b) in a sectional view along line V-V,

FIG. 5, a unit operation station of the bioprocessing system of FIG. 1 during the standard routine in a sequence a) to e),

FIG. 6, the unit operation station of FIG. 5 a) during a connecting process and b) during a disconnecting process,

FIG. 7, the unit operation station of FIG. 5 during the transport of a cartridge from the drive location to the cartridge waste storage in a sequence a) to b),

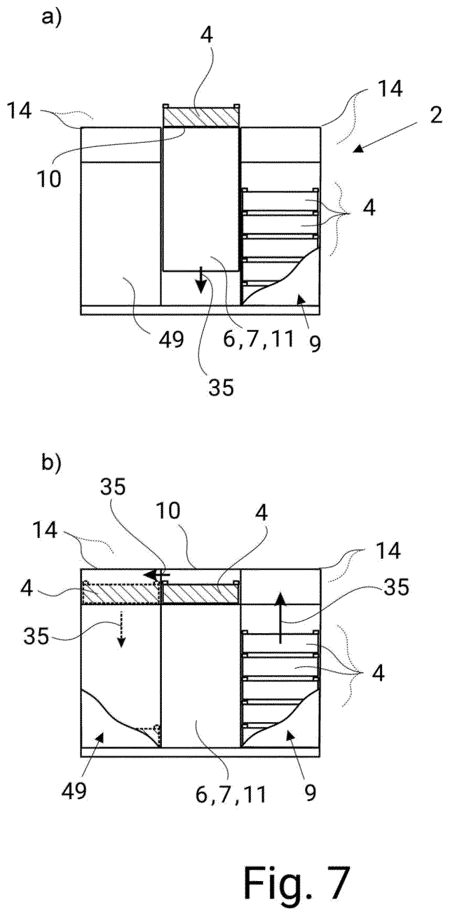

FIG. 8, the closed connection process in a schematic representation in a sequence a) to e),

FIG. 9, examples of cartridges and

FIG. 10, a depiction of a functional interface of the cartridges and a container connectable to the functional interface.

### DETAILED DESCRIPTION

The integrated bioprocessing system 1 shown in the drawings can be adapted to perform a bioprocess for the manufacturing of genetically modified T cells. Here, T cells are genetically modified to express a chimeric antigen receptor (CAR). Consequently, the term “CAR-T cells” describes T cells that have been genetically modified to express a CAR. The genetically modified CAR-T cells, which represent the product of the bioprocess, may be administered to a patient and used to start or resume cancer treatment in the patient. As the bioprocess is performed, the initial immune cell culture is gradually processed. All explanations given are mainly directed to such a bioprocess. It may be pointed out, however, that those explanations are fully applicable to other bioprocesses as well.

The term “liquid immune cell culture” is to be understood in a broad sense and refers to an immune cell culture comprising at least one type of immune cells suspended as particles in any type of liquid. As will be explained below, the liquid immune cell culture may comprise other cell types that are not immune cells. Hence, the term “liquid immune cell culture” refers to a liquid immune cell culture at any stage during the bioprocess. Consequently, the type and fraction of immune cells present in the liquid immune cell culture will change during the bioprocess applied as certain immune cells are enriched or depleted from the liquid immune cell culture and/or the immune cells are genetically modified.

The term “immune cells” generally refers to different types of white blood cells. Hence, the term “immune cells” includes a variety of cells, for example, but not limited to dendritic cells, T lymphocytes, also referred to as T cells, B lymphocytes, natural killer cells, macrophages or the like. Immune cells may also include subtypes of immune cells, for example tumor-infiltrating lymphocytes or different types of T cells. Subtypes of a certain type of immune cells may be classified based on the type of antigen present at the cell surface. Hence, the term immune cells may for example refer to T cells comprising the surface antigen CD4 (“CD4+ T cells”). Typically, a certain type of immune cells, e.g., T cells, in some embodiments a certain subtype of immune cells, e.g., CD4+ T cells, will be selectively enriched by the bioprocess, while other immune cells, e.g. macrophages, and/or other cell types that are not immune cells, e.g., erythrocytes, and/or other subtypes of immune cells, e.g., CD8+ T cells, will be depleted from the liquid immune cell culture. The immune cells to be enriched are referred to as target immune cells, all other components to be depleted from the liquid immune cell culture are referred to “impurities”. Further, and as mentioned above, the target immune cells might be genetically modified.

The term “naive cells” refers to cells that can still differentiate into different target cell types. In particular, stem cells and their derivatives prior to full differentiation into a specific cell type are naive cells. The term also comprises naive immune cells.

The term “liquid” is to be understood in a broad sense as well and refers to any liquid and/or particle-containing liquid that is processed within the integrated bioprocessing system 1. Hence, the term liquid might refer to media, including buffers for washing and feed media for cell expansion, waste, the liquid immune cell culture, byproducts obtained during the bioprocess, samples and/or an initial immune cell culture.

The term “waste” refers to any liquid and/or particle-containing liquid obtained during the bioprocess, wherein the respective liquid and/or particle-containing liquid may be discarded and is not used further. However, waste may be stored and, in some instances, be used again before being discarded. For example, if the number of T-Cells extracted from the cell culture is too low, more T-Cells may be extracted from the waste.

The term “initial immune cell culture” refers to a liquid immune cell culture before a first processing step of the bioprocess is applied. The initial immune cell culture might be derived from different sources. In an approach often referred to as “autologous cell therapy”, the initial immune cell culture is obtained from a donor, who is also the recipient of the product after the bioprocess has been performed completely. In “allogenic cell therapy”, the initial immune cell culture might be derived from at least one donor, who is not the recipient of the product. The initial immune cell culture might be derived from more than one donor and/or used for more than one recipient. In this case, the immune cell cultures obtained from different donors are combined into a single initial immune cell culture.

In various embodiments, the initial immune cell culture is obtained in a process called “leukapharesis”. In leukapharesis, immune cells are obtained from the patient. Additionally or alternatively, the initial immune cell culture might also be obtained from a tissue of the patient. As mentioned above, the initial immune cell culture might also be obtained by one or more donors that are not the patient. Additionally, in some cases, other cells may be obtained from the same patient, for example, tumour cells, which are used within the bioprocess to modulate the behaviour of the immune cells.

Depending on the source of the initial immune cell culture and the bioprocess to be carried out, the initial immune cell culture, particularly the type, amount and distribution of impurities as well as target immune cells might vary. Further, the initial immune cell culture might also be obtained from a frozen source or the initial immune cell culture may be frozen and thawed when needed.

Presently, it can be that any initial immune cell culture used in the bioprocess is used for only a single patient or a small number of patients, for example up to ten patients. If that is not the case then the bioprocess presently described will yield only a cell culture for a single patient or a small number of patients and cell cultures for other patients are derived from different bioprocesses. Therefore, the proposed integrated bioprocessing system 1 is used for small scale bioprocesses. The bioprocessing system 1 itself may be of larger scale by performing multiple bioprocesses in parallel.

The term “processing step” refers to a distinctive step that is being performed as part of a bioprocess involving a liquid immune cell culture. The type and sequence of processing steps performed depends on the bioprocess that is carried out on the respective liquid immune cell culture and on the type of immune cell culture. Depending on the mentioned parameters, different processing steps may be combined in any given sequence. Additionally or alternatively, the configuration of a processing step might differ, and/or processing steps may be repeated and/or omitted. Each processing step comprises at least one operation. An operation is the smallest unit of steps in a bioprocess that has a defined beginning and end. Usually, a processing step comprises several operations like pumping, mixing, centrifuging and the like. If one or more operations of a processing step are described herein, it can be that those comprise most or all of the relevant operations of the respective processing step.

Proposed is a method for performing a bioprocess on liquid immune or naive cell cultures to obtain processed cell cultures. The processed cell cultures may be used for a cancer therapy or the like as described above. The method may also include performing multiple bioprocesses on multiple liquid immune or naive cell cultures to obtain multiple processed cell cultures. In various embodiments, the processed cell cultures are destined for autologous or allogenic cell therapy. Each bioprocess is performed on different cell cultures, all processing steps performed on one cell culture are to be understood as a single bioprocess. The multiple cell cultures can be used to treat a patient. Alternatively, multiple cell cultures can be obtained from a single body of input patient material to evaluate and compare different production processes. The production processes that are identified as optimal would then be used for subsequent therapeutic manufacture.

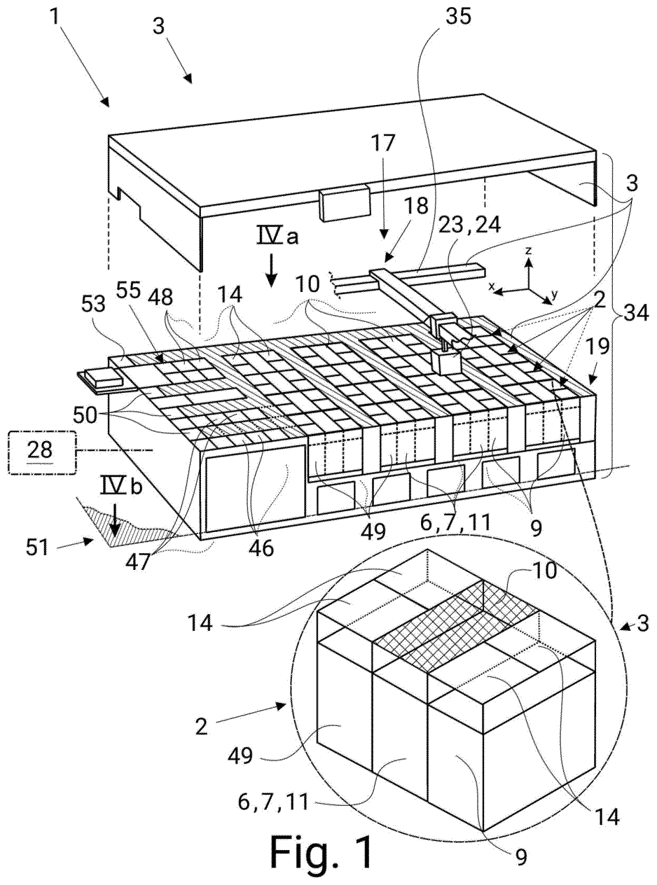

The bioprocess is performed on an integrated bioprocessing system 1, for example the one shown in FIG. 1. The integrated bioprocess system 1 in FIG. 1 is merely an exemplary embodiment and other embodiments may differ significantly as will be clear from the following description.

The bioprocess comprises a sequence of processing steps, wherein the processing steps each comprise at least one operation. In the following, unit operations will also be mentioned. A unit operation is the sum of operations performed at a unit operation station 2 present in some embodiments. A unit operation may comprise part of a processing step or even multiple processing steps.

The bioprocess system 1 comprises a base structure 3 and preconfigurable cartridges 4. The cartridges 4 may be preconfigured with a cartridge fluidic structure 5 with respect to at least one operation. The preconfiguration can be done outside the bioprocess system 1. Based on this, depending on the configuration of the cartridge fluidic structure 5, just about any operation may be realized with the preconfigured cartridge 4. This way, each cartridge fluidic structure 5 may be highly individualized for different bioprocesses and with that particularly for different liquid cell cultures as well.

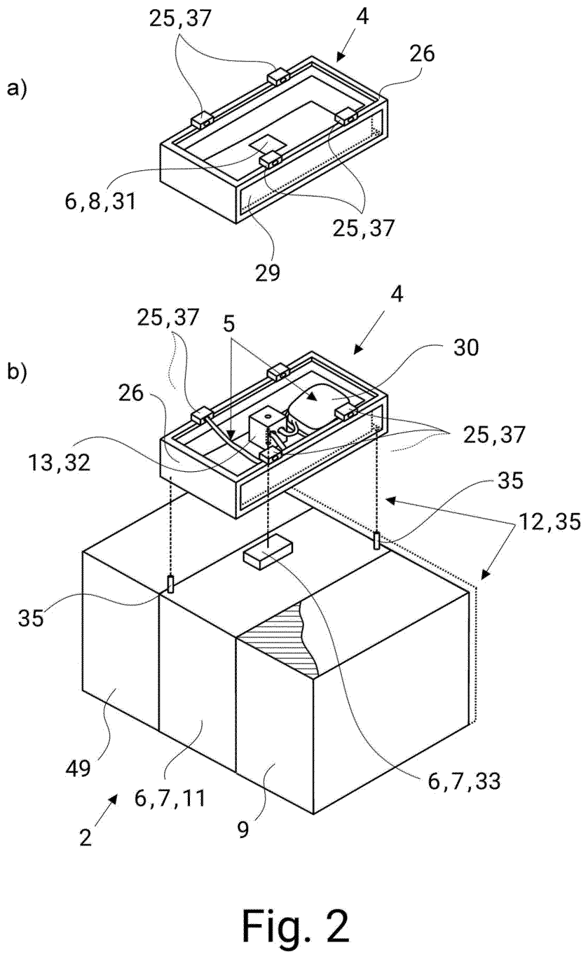

FIG. 2a shows a cartridge 4 in its not yet configured state without a cartridge fluidic structure 5, while FIG. 2b shows a cartridge 4, that has been preconfigured with a cartridge fluidic structure 5 with respect to at least one operation.

The bioprocess system 1 performs operations of the bioprocess via an interaction of the base structure 3 with the cartridges 4. That is, the base structure 3 forms the basis for performing the bioprocess and the cartridges 4 form the basis for flexibly performing different bioprocesses. The cartridges 4 and the base structure 3 comprise matching standardized interfaces 6 for an interaction of the base structure 3 with the respective cartridge 4. Due to the standardization, the flexibility is achieved without having to adapt the base structure 3 to the different operations.

Therefore, the bioprocess system 1 performs at least two operations of the bioprocesses inside at least two differently preconfigured cartridges 4 by an interaction of the base structure 3 with the cartridges 4 via the same base structure interface 7 and/or identical base structure interfaces 7 and matching cartridge interfaces 8. It is possible to use the same base structure interface 7 for the two operations but not mandatory. The cartridge interfaces 8 may differ as long as they are compatible to the base structure interface 7. For example, a cartridge 4 may simply have a hole in a place where a mechanical energy transmitting element may enter if the cartridge 4 in its preconfiguration does not need any mechanical energy.

The term “interface” includes all functional, at least partially mechanical, connections between the base structure 3 and the cartridge 4. There is at least the base structure interface 7 that supports the at least two operations. The base structure 3 may comprise multiple base structure interfaces 7 for performing the operations inside of cartridges 4. These base structure interfaces 7 directly influence the actual operation. The base structure interface 7 may comprise an electrical connection providing electrical energy to the cartridge 4 and/or a cable-bound signal connection providing signals to the cartridge 4 and/or a mechanical connection providing mechanical energy to the cartridge 4 and/or a pneumatic connection providing pneumatic energy to the cartridge 4.

The base structure 3 may comprise multiple different base structure interfaces 7 for performing operations of different subsets of processing steps. In various embodiments, those base structure interfaces 7 are at least partially identically to each other, for example comprise the same electrical and/or mechanical and/or pneumatic and/or signal connector. They may however comprise further distinguishing elements such that not every operation can be performed by every base structure interface 7. In particular, functional elements may be present as part of the base structure interface 7 only at some locations and the base structure interface may be adapted to the functional interface.

There may be different additional base structure interfaces for auxiliary functions in particular the standardized transport and placement interface 35. The transport and placement interface 35 here and in some embodiments comprises one or more transport elements that transport cartridges 4 and/or receptacles 15 and/or containers 30 between base structure interfaces 7.

Other than the described bioprocess steps may be part of the bioprocesses and performed by the bioprocess system 1.

In various embodiments, a bioprocess is performed in at least three, or at least four differently individualized cartridges 4 by the bioprocessing system 1. In various embodiments, no more than 8 differently individualized cartridges 4 are used in a single bioprocess.

The plural term “cell cultures” also comprises what might be named “cell culture” in singular, too.

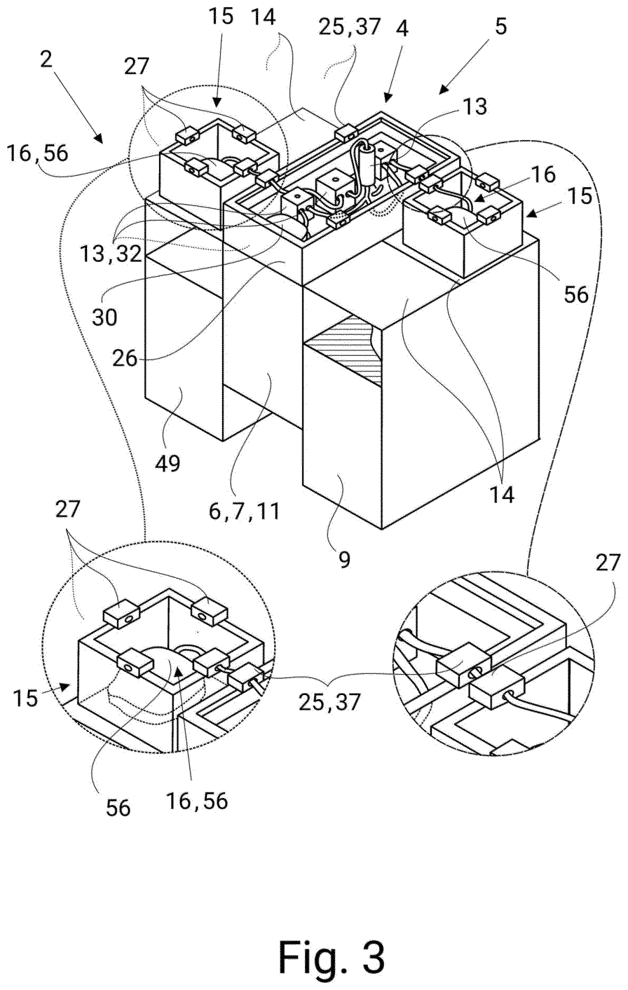

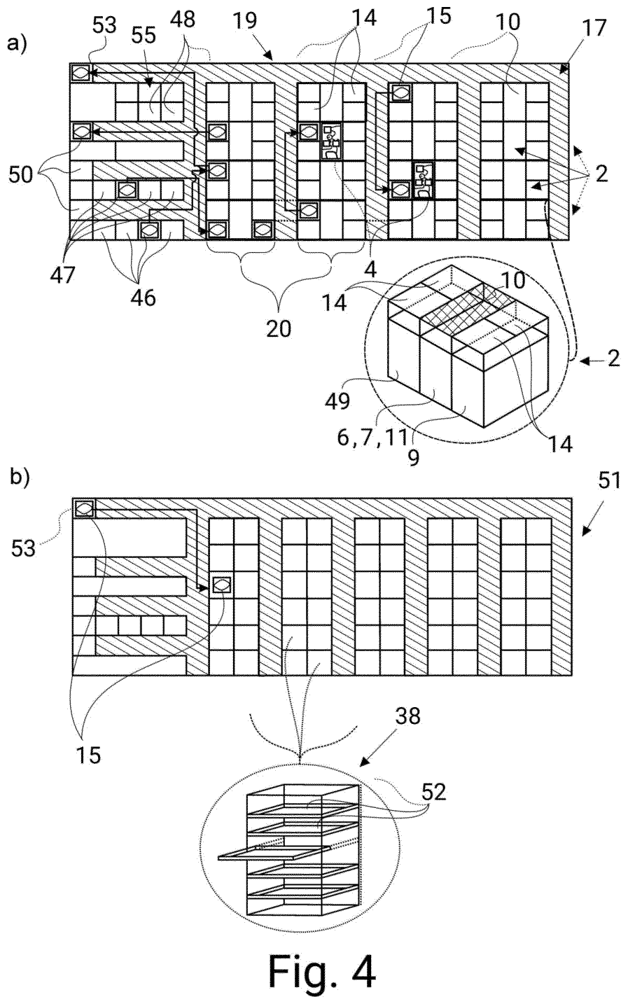

The embodiment shown in the drawings will now be explained before further variations will be discussed which are not all shown in the drawings. FIG. 1 shows the integrated bioprocessing system 1, FIG. 2 shows a unit operation station 2 which is one way of flexibly providing a main part of the bioprocessing system 1. FIG. 3 shows the performance of a unit operation on the unit operation station 2. FIG. 4 shows how the bioprocessing system 1 can be built up from multiple such unit operation stations 2. The unit operation stations 2 together with other components like a transport mechanism build the base structure 3 and interact with the cartridges 4.

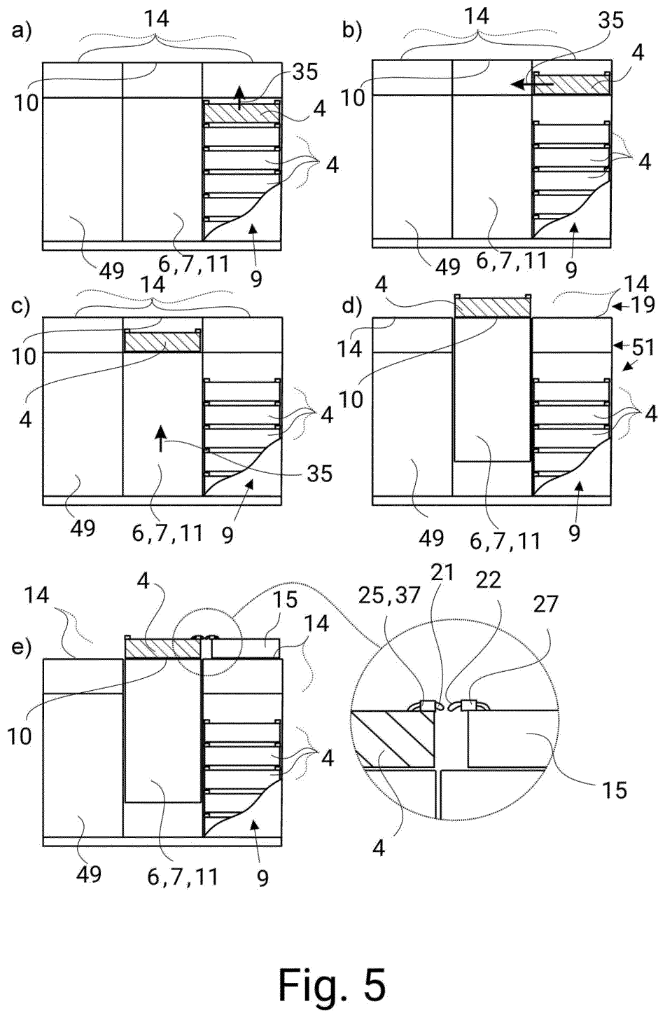

In the embodiment shown in the drawings, for performing one or more operations, a standard routine is defined. According to the standard routine, one of the cartridges 4 is being transported from a cartridge storage unit 9 of a unit operation station 2 to a drive location 10 of a cartridge drive unit 11 of the unit operation station 2 (sequence FIGS. 5a), 5b), 5c), 5d)) by a local transport mechanism 12 of the unit operation station 2, which is only indicated in FIG. 2b. This local transport mechanism 12 may for example comprise a linear actuator and/or several linear actuators and/or a combination of a linear actuator and a multi-axis robotic manipulator, a conveyor belt or the like. Subsequently, the cartridge 4 is being brought into operative coupling with a cartridge drive unit 11. Here, the cartridge drive unit 11 is a standardized interface 6 between the base structure 3 comprising the unit operation station 2 and the cartridge 4.

In various embodiments, performing of each and any one of the unit operations includes performing the above noted standard routine, which provides the transport of the respective cartridge 4 from the cartridge storage unit 9 to the cartridge drive unit 11 and the operative coupling of the cartridge 4 with the cartridge drive unit 11. The subsequent performance of the unit operation is then individualized dependent on the preconfiguration of the cartridge 4. Neither the unit operation stations 2 nor the unit operations are necessarily present in every embodiment.

The operative coupling between the cartridge 4 and the cartridge drive unit 11 makes it possible for the cartridge 4 to be a completely passive component, without any kind of actuators. Via this operative coupling, any actuation may be transmitted from the cartridge drive unit 11 to the cartridge 4. However, it is generally possible, that the cartridge 4 comprises additional actuators.

The term “operative coupling” is to be understood in a broad way. In various embodiments, it means the functional engagement of the cartridge drive unit 11 to the cartridge 4. Hence, the term “coupling” may refer to the physical engagement between both components. Additionally or alternatively, it may also refer to an indirect connection (e.g., by magnetic actuation) established between two components. It may be the case that the cartridge drive unit 11 actuates a functional device 13 of the cartridge 4. All of this is performed via the base structure interface 7 and the cartridge interface 8.

According to various embodiments, that at least two of the cartridges 4, in some embodiments all cartridges 4, are identical to each other with the exception of the respective cartridge fluidic structures 5, which may be configured to the respective unit operations. This means, that as far as the respective cartridge fluidic structure 5 is concerned, the cartridges 4 may (but do not have to) deviate from each other. With the identical cartridges 4 in this sense, not only the transport of the cartridges 4 may be standardized, but also the transfer of liquids to and from the cartridges 4.

According to another embodiment it is proposed, that the at least one unit operation station 2 comprises at least one transfer location 14, indicated in FIG. 3. In various embodiments, the transfer location 14 is provided by a surface or a pad for receiving a receptacle 15. Accordingly, receptacles 15, that have been preconfigured with a receptacle fluidic structure 16 for containing a liquid, such as an immune or naive cell culture, are being transported by a global transport mechanism 17, depicted in FIG. 1, to a transfer location 14, in order to transfer liquid between a cartridge 4, which has been transported to the drive location 10, and the receptacle 15 and/or to perform a respective unit operation on the liquid contained in the receptacle 15.

In various embodiments, each unit operation station 2 comprises four transfer locations 14. As shown in FIG. 1 and in various embodiments, each transfer location 14 is of a rectangular geometry, with two transfer locations 14 being located next to each other. In various embodiments, two pairs of transfer locations 14 are located opposite each other with the drive location 10 located between each pair of transfer locations 14.

As will be explained below, the receptacles 15 located in the transfer locations 14 need to be aligned to the cartridge 4 located in the drive location 10. For alignment, either the global transport mechanism 17, such as a robotic mechanism 18 may be used, or the transfer location 14 may comprise a positioning mechanism to align the receptacle 15 to the cartridge 4.

According to one embodiment it is proposed, that at least two of the receptacles 15, in some embodiments all receptacles 15, are identical to each other with the exception of the respective receptacle fluidic structures 16, which may be configured to the respective liquid to be contained. This means, that as far as the respective receptacle fluidic structure 16 is concerned, the receptacles 15 may (but do not have to) deviate from each other. The liquid to be contained in the receptacle fluidic structure 16 may depend on the process step being performed. This means, that the complete handling of the receptacles 15 including transport, establishing a fluid connection etc. may be standardized for all receptacles 15. An example for those identical receptacles 15 is shown in FIG. 3.

According to various embodiments it is proposed, that the integrated bioprocessing system 1 comprises a number of unit operation stations 2, in some embodiments, that the transfer locations 14 of the unit operation stations 2 are arranged in a first plane 19 and that the receptacles 15 are being transported by the global transport mechanism 17 to and from the transfer locations 14 in this first plane 19. The first plane 19 is here and in some embodiments aligned horizontally. By arranging the transfer locations 14 in the first plane 19, transport of the receptacles 15 to and from the transfer locations 14 is simplified. In addition, and as will be explained below, the receptacles 15 are then located in the same plane as the cartridge 4 that is located in the drive location 10.

In various embodiments, at least part of the number of unit operation stations 2 are arranged in unit operation station slices 20. Each unit operation station slice 20 can include separate transport devices such as wheels or the like to move the unit operation station slices 20 into and out of the integrated bioprocessing system 1. By being able to remove a unit operation station slice 20 including the number of unit operation stations 2 located within that unit operation station slice 20 from the integrated bioprocessing system 1, servicing of the unit operation stations 2, which will be explained further below, is particularly simple. Further, a unit operation station slice 20 may be removed from or introduced into the integrated bioprocessing system 1 without affecting the operability of the remaining unit operation stations 2 within the integrated bioprocessing system 1. This is particularly advantageous, if a part of the integrated bioprocessing system 1 needs to be accessed for maintenance.

According to one embodiment it is proposed, that the at least one bioprocess is a closed bioprocess, such that all liquids involved in the respective bioprocess are being kept within a closed inner volume. In the shown embodiment, this closed structure is realized by performing at least part of the liquid handling involved in the respective bioprocess within tubes.

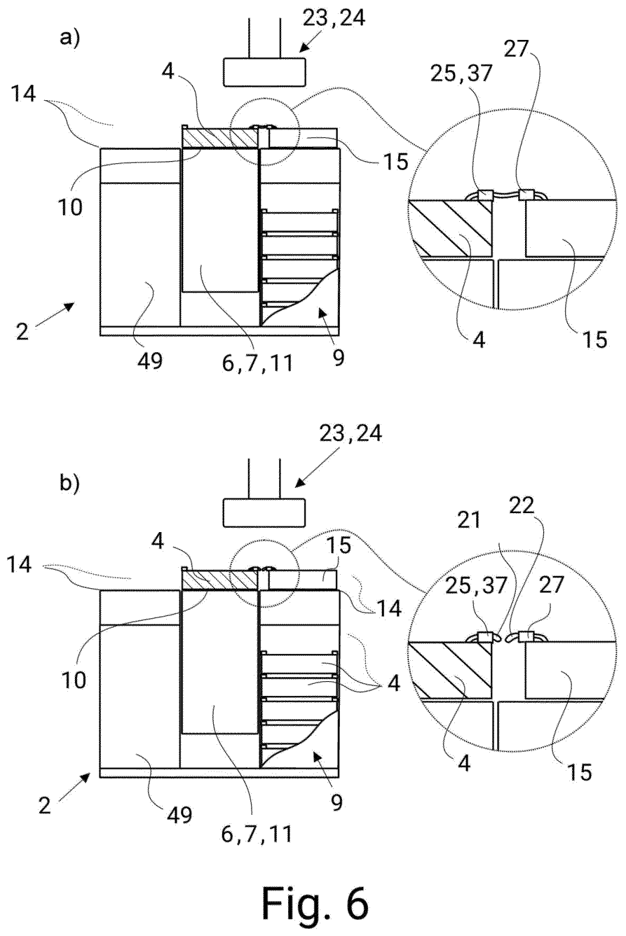

For an easy realization of a closed bioprocess it is proposed, that for the transfer of liquid between the cartridge 4 and the receptacle 15, a cartridge transfer tube 21 of the cartridge 4 and a receptacle transfer tube 22 of the receptacle 15 are being connected in a closed connection process by a tube connection system 23. This is indicated in FIG. 6a. In various embodiments, the connection process is being performed by a tube connection system, particularly by a tube welding system 24.

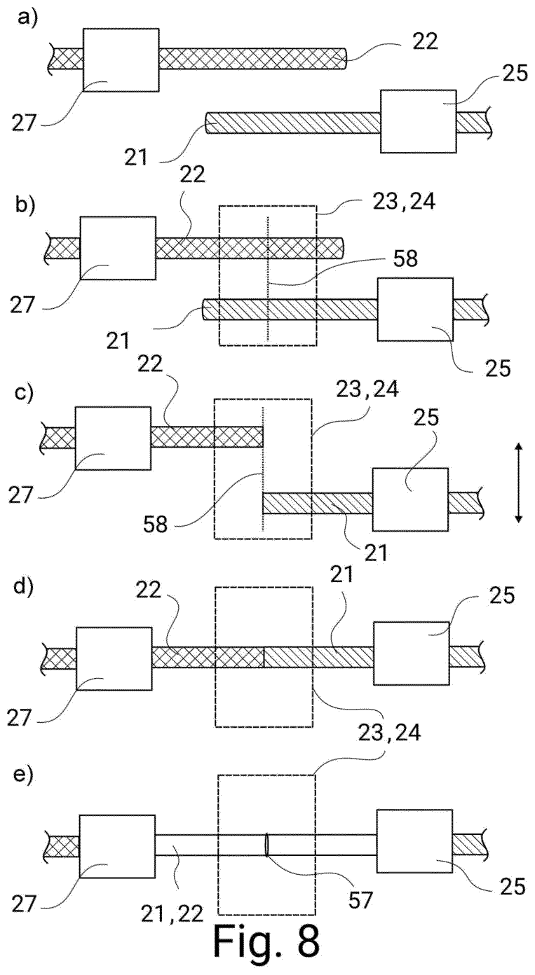

The connection process is shown in FIG. 8. The connection process is performed by the tube connection system 23, in particular by a tube handling device (not shown) of the tube connection system 23. It can also include a step of arranging the tubes to be connected relative to the rest of the tube connection system 23 (sequence FIG. 8a) to FIG. 8b)) and a subsequent step of welding the tubes to be connected by the tube connection system 23 (FIG. 8d)). After welding, and as depicted in FIG. 8e), the tubes are connected at a weld location 57. Further in various embodiments, before the step of welding, a step of trimming the tubes to be connected is provided within the connection process (FIG. 8b)). In various embodiments, and as depicted in FIG. 8c), trimming of the tubes is performed by a blade 58.

As depicted in FIG. 3, the cartridge transfer tube 21 and the receptacle transfer tube 22 are provided by the cartridge fluidic structure 5 and the receptacle fluidic structure 16. In various embodiments, the cartridge fluidic structure 5 and the receptacle fluidic structure 16 have been preconfigured in such a way that each cartridge transfer tube 21 is located in a predefined cartridge anchor point 25 provided by a cartridge frame 26 and that each receptacle transfer tube 22 is located in a predefined receptacle anchor point 27 provided by the receptacle 15. This is shown in FIG. 3, for example. In various embodiments, the cartridge anchor points 25 and the receptacle anchor points 27 are arranged in such a way that one cartridge anchor point 25 faces one receptacle anchor point 27, when the respective cartridge 4 is in the drive location 10 and the respective receptacle 15 is in the transfer location 14.

In various embodiments, and as depicted in FIG. 3, the cartridge transfer tube 21 protrudes out of the cartridge anchor point 25 towards the transfer location 14 that the receptacle 15 to be connected is located in. It can also be that the receptacle transfer tube 22 protrudes out of the receptacle anchor point 27 towards the cartridge anchor point 25 that the cartridge transfer tube 21 is located in. A welding system 24 connects the receptacle transfer tube 22 and the cartridge transfer tube 21 in the closed connection process.

In addition, as indicated in FIG. 6b, the welding system 24 can be designed to perform a disconnecting process as well. In various embodiments, the transfer tubes to be disconnected are closed and cut at their respective ends, such that the inner volumes of the tubes remain sterile. Here it may also be pointed out, that the use of such a welding system 24 enables to perform multiple welds subsequently on the same transfer tube. This provides flexibility as for example the same receptacle transfer tube 22 may be subsequently connected to different cartridge transfer tubes 21.

According to one embodiment it is proposed, that at least two said bioprocesses are being performed at least partly simultaneously by the integrated bioprocessing system 1, in various embodiments, as coordinated by an electronic process control 28. This is indicated in FIG. 4a with two cartridges 4 of two separate unit operation stations 2 having been transported to a respective drive location 10.

In this shown embodiment, the cartridge 4 comprises a cartridge carrier 29, which receives the components of the cartridge fluidic structure 5. The advantage is that the cartridge carrier 29 provides a standardized structure to receive the cartridge fluidic structure 5. Hence, the size of the cartridge carriers 29 can be identical for all unit operation stations 2 and, thus, independent of the specific unit operation performed at a unit operation station 2. Accordingly, the cartridge fluidic structure 5 can be arranged as desired within the cartridge carrier 29 while the cartridge carrier 29 provides a standardized interface 6 to the cartridge frame 26 and to the cartridge drive unit 11. Additionally, the cartridge carrier 29 is out of contact with any liquids handled at the unit operation station 2. Hence, the cartridge carrier 29 can be reused after the cartridge 4 has been used in a unit operation. Further, using a cartridge carrier 29 to receive the components of the cartridge fluidic structure 5 ensures that any liquid that may leak from the cartridge fluidic structure 5, for example in case of a breach in the cartridge liquid container 30, is contained within the cartridge 4 and does not contaminate other elements, for example the cartridge drive unit 11 of the integrated bioprocessing system 1.

As depicted in FIG. 2b, the cartridge carrier 29 may comprise drive recesses 31 to enable the drive structure of the cartridge drive unit 11 to engage with components of the cartridge fluidic structure 5. For example, and as depicted in FIG. 2a, the cartridge carrier 29 may comprise a drive recess 31 at a position, a pump head of a peristaltic pump 32 is to be placed. For actuation of the components of the cartridge fluidic structure 5 by the interface provided by the base structure 3, it is important that the drive recesses 31 in the cartridge carrier 29 are aligned to the respective interface of the base structure 3.

In various embodiments, the material of the cartridge carrier 29 is a multi-use material like plastic or the like. Further, and as mentioned above, each cartridge carrier 29 can be standardized regarding its size. It can also be that the dimensions of the cartridge carrier 29 regarding its longitudinal and transverse direction correspond to the cartridge drive unit 11. As will be explained below, the cartridge drive structure 33 of the cartridge drive unit 11 provides interfaces that engage with the components of the cartridge fluidic structure 5.

As can be seen, the base structure 3 can be composed by multiple, even exchangeable, components. In various embodiments, during use of the bioprocessing system 1, the base structure 3 is an interconnected structure. The base structure 3 is not composed by unrelated and unconnected components.

All liquids involved in the bioprocess are here and in some embodiments being kept within one or more closed inner volumes. This ensures that the sterility of all liquids is maintained. It further ensures that no cross-contamination between different cell cultures occurs. This is especially important when multiple cell cultures are processed in parallel. Further, the requirements of the environment regarding sterility are largely reduced. The term “closed inner volume” presently means, that all noted liquids are being maintained and guided within a volume, that is separated from atmosphere. The term “atmosphere” presently represents the volume outside the above noted, closed inner volume. This ensures to maintain sterility within the closed inner volume. As explained above, maintaining sterility is a critical aspect in the manufacturing process. The closed inner volume may easily be realized, if at least part of the liquid handling involved in the respective unit operation is being performed within tubes. In various embodiments, the integrated bioprocessing system 1 comprises an enclosure 34 in which the bioprocess is performed. Inside the enclosure 34 a sterile atmosphere may be provided, however, in various embodiments, it is not as sterile as the closed inner volumes.

According to one embodiment it is proposed, that the two operations of the bioprocess are operations of different processing steps, and/or, that the bioprocessing system 1 performs at least two, at least three, or at least four, operations that are part of different bioprocesses in parallel in identically preconfigured cartridges 4.

Additionally or alternatively, the bioprocessing system 1 may perform at least two, at least three, or at least four, operations that are part of different bioprocesses, in particular part of different processing steps of the different bioprocesses, in parallel in differently preconfigured cartridges 4.

According to one embodiment it is proposed, that the bioprocessing system 1 performs at least two bioprocesses, at least three bioprocesses, at least five bioprocesses, or at least ten bioprocesses, each comprising at least two, at least three, or at least four different processing steps, in particular in parallel, wherein the bioprocessing system 1 performs at least one operation per processing steps, wherein the bioprocessing system 1 performs those operations in at least two, at least three, or at least four, differently preconfigured cartridges 4 per bioprocess. In various embodiments, all of these operations are automatically performed without a manual change of the base structure 3.

According to one embodiment it is proposed, that the operations comprise operations of the processing step or processing steps “enrichment” and/or “selection” and/or “activation” and/or “loading” and/or “genetic modification” and/or “expansion” and/or “formulation” and/or “fill” and/or “wash” and/or “separation”, and/or, that the processing steps comprise the processing steps “enrichment” and/or “selection” and/or “activation” and/or “loading” and/or “genetic modification” and/or “expansion” and/or “formulation” and/or “fill” and/or “wash”and/or “separation”.

Details of these processing steps will be given below. All explanations given with regard to any feature or method step described herein may apply to any one, any combination of or all of the named bioprocess steps and/or bioprocess operations. It is one advantage of the proposed system that it can be adapted to different processing steps as needed. It is a further advantage of the proposed system that the adaptations to different processing steps may be made while the bioprocess is already being performed. In particular, changes to the order of the processing steps may be made and/or processing steps may be repeated.

In various embodiments, the processing steps “enrichment” and/or “selection” and/or “activation” and/or “loading” and/or “genetic modification” and/or “expansion” and/or “formulation” and/or “fill” and/or “wash” and/or “separation” can be performed and/or repeated in any order automatically without manual intervention as part of one bioprocess and by transferring the cell cultures between cartridges 4 as needed. Anyway, in various embodiments, at least one transfer of a cell culture from one cartridge 4 to another can be performed fully automatically.

Turning now to the cartridges 4 from a general point of view, it may be the case that the bioprocessing system 1 comprises single step cartridges 4, whereby the bioprocessing system 1 performs only operations of a single processing step inside the single step cartridges 4. The less processing steps are combined in cartridges 4, the easier the flexible combination and repetition of steps becomes.

Additionally or alternatively, the bioprocessing system 1 may comprise shared cartridges 4, whereby the bioprocessing system 1 performs operations of, in particular consecutive, processing steps inside the shared cartridges 4. This reduces the number of transport steps between operations.

Operations of the processing steps “enrichment” and/or “selection” and/or “activation” and/or “loading” and/or “genetic modification” and/or “formulation” and/or “fill” and/or “wash” and/or “separation”may be performed in a single step cartridge 4.

As already hinted, it may be the case that the preconfigured cartridges 4 comprise a preconfigured cartridge fluidic structure 5, and/or, that the preconfigured cartridges 4 comprise a, in particular preconfigured, functional device 13 for the operation performed inside the cartridge 4, and/or, that at least some, in some embodiments all, cartridges 4 used for the operations are identical prior to being preconfigured. A preconfigured functional device 13 is a functional device 13 that was added to the cartridge as part of the preconfiguration. The functional device 13 itself may have a standard configuration.

The cartridges 4 without their preconfigured content can be seen as an adapter between the base structure 3 providing energy and organization and the functional devices 13 and the fluidic structures together performing the actual bioprocess. This abstraction layer allows commonality at the level of the base structure and flexibility at the level of the cartridges.

As has already been explained with view to various embodiments of the unit operation station 2, it may be the case that the standardized base structure interface 7 comprises an active, energy transmitting interface, that the base structure 3 via the active base structure interface 7 and the matching cartridge interface 8 transfers mechanical and/or pneumatic and/or electrical energy to the cartridge 4, in particular drives a functional device 13 of the cartridge 4 via the transferred energy. In various embodiments the interfaces comprise a plug system between the base structure 3 and the cartridges 4. The plug may comprise several connectors for electrical energy and signals. Further, a plug for a mechanical drive protruding from the base structure 3, in particular the cartridge drive unit 11, may be provided on the cartridge 4.

Additionally the base structure 3 may comprise a transport and placement interface 35, which would here be the top of the unit operation station 2 together with the global transport mechanism 17, the transfer location 14 and so on. Then, the base structure 3 via the base structure interface 7 and the matching cartridge interface 8 transports the cartridge 4 into a position in which the bioprocessing system 1 performs at least one of the operations inside the cartridge 4 and holds the cartridge 4 in the position.

It has been explained how the cartridges 4 may comprise functional devices 13. However, some or all functional devices 13 may be multi-use components and providing a large number of those in the cartridges 4 may not be efficient.

According to one embodiment it is proposed, that the bioprocessing system 1, in particular the base structure 3, comprises functional devices 13, in various embodiments, that functional devices 13 of the base structure 3 are fixed in place next to dedicated standardized interfaces 6 for one or more operations performed by the functional device 13 via a, in particular standardized, function interface of the functional device 13, and function receiving interfaces of the cartridges 4, in some embodiments standardized for different functional devices 13 of different processing steps, or, that functional devices 13 of the base structure 3 are transported into place next to non-dedicated standardized interfaces 6, that the bioprocess structure performs one or more operations by the functional device 13 via a, in particular standardized, function interface of the functional device 13, and function receiving interfaces of the cartridges 4, in some embodiments standardized for different functional devices 13 of different processing steps.

The functional interfaces may be dedicated interfaces if necessary. All cartridges 4 may have one or more dedicated interfaces which are not always used dependent on the respective use of the cartridge 4. However, it may also be the case that the cartridges 4 comprise one or more standardized functional interfaces that are not dedicated to a single operation or processing step. For example, different processing steps may comprise applying contactless energy onto a dedicated cartridge fluidic structure 5 and/or a functional device 13. The cartridge fluidic structure 5 and/or the functional device 13 may be placed in a standardized place of the cartridge 4 and comprise further components, like a reflector yet to be explained, to receive the contactless energy in a standardized manner through an interface which can for example be a hole of a certain dimension in the cartridge 4 through which the contactless energy emitting device is brought into close contact or vicinity to the individual cartridge fluidic structure 5 and/or the functional device 13. The cartridge fluidic structure 5 can comprise rigid tubes or the like which may be single use and connect to the functional device 13 to receive the contactless energy. A standardized function interface may be the standardized interface 6.

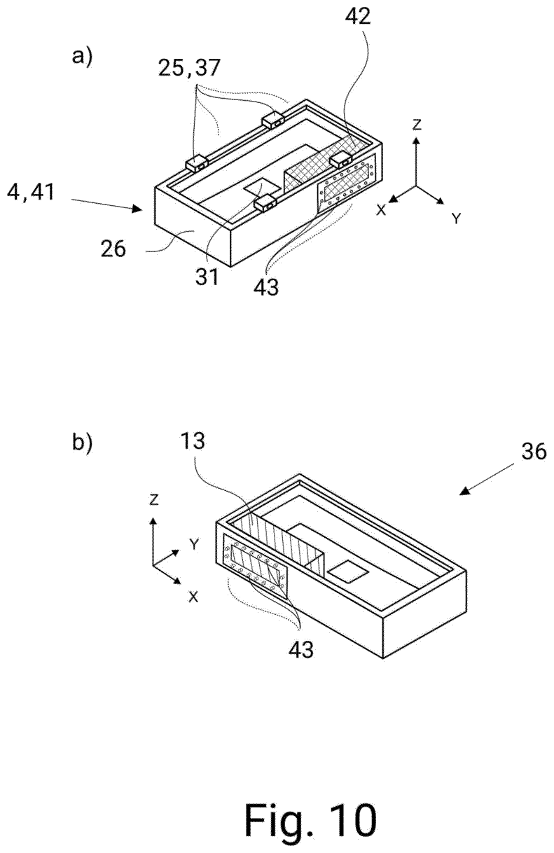

As depicted in FIG. 10b), the functional devices 13 may also be provided in containers 36 which are standardized for their interaction with the cartridges 4 and/or the base structure 3, in some embodiments such that the base structure 3 does not need to know what the content is to handle the container 36. The container 36 and/or the receptacles 15 and/or the cartridges 4 may have a standard size or standard sizes and/or a standard transport interface. The containers 36 and/or receptacles 15 may have a standard interface 6 for connection to the cartridges 4 and/or to the base structure 3.

Every, in particular yet to be described, functional device 13 or combination of functional devices 13 of the base structure 3 may be stationary or placed inside a container 36.

According to one embodiment it is proposed, that the bioprocessing system 1 performs the operations inside of cartridges 4 with interfaces identical between the cartridges 4 for receiving the cell cultures, in particular from receptacle liquid containers 56, and/or for forwarding the cell cultures out of the cartridge 4 after the operation, in particular to receptacle liquid containers 56 of receptacles 15, and/or for receiving consumables, in particular from receptacles 15. It can be seen well in FIGS. 2 and 3 how a single type of interface can be used to connect a cartridge 4 to different receptacles 15. While not shown, it is also clear that instead of a receptacle 15 in FIG. 3, another cartridge 4 can be placed next to the shown cartridge 4. Again, the same interface can be used to transfer fluid between the receptacles 15 and/or cartridges 4. Here, the interface between a cartridge 4 and a receptacle 15 comprises a single tube connection. The interface between two cartridges 4 would here comprise one or two tube connections. When looking at FIG. 3 it is also clear that the receptacles 15 connected to the cartridge 4 can further be connected to other receptacles 15, those being connected to the same cartridge 4 or a different cartridge 4 or no cartridge.

In another embodiment, the base structure 3 and/or a receptacle 15 and/or a cartridge 4 may comprise a pump for pumping liquid through the interface. In particular, the pump can be part of the base structure 3 and provided by the base structure 3 for the interface between cartridges 4 and cartridges 4, cartridges 4 and receptacles 15 or receptacles 15 and receptacles 15. This way, a certain number of pumps can be used in the base structure 3 as multi-use components with varying cartridges 4 and receptacles 15. There may also be a type of standard pump for the interfaces and other pumps inside the cartridges 4 as needed. In one embodiment, a pump is part of the base structure 3 and of the standardized base structure interface 7. It may protrude into the cartridge 4 in a connected state or otherwise act on the cartridge fluidic structure 5.

Additionally or alternatively the bioprocessing system 1 may perform the operations inside of cartridges 4 with interfaces identical between the cartridges 4 and/or for receiving mechanical and/or pneumatic and/or electrical energy and/or cable-bound signals from the base structure 3 and or for receiving energy from a functional device 13 and/or providing the cell cultures to the functional device 13.

Generally, the cartridges 4 may comprise one or more fluid interfaces 37. In various embodiments, the interface or interfaces for receiving the cell cultures and/or for forwarding the cell cultures and/or for receiving the consumables and/or for providing the cell cultures to functional devices 13 is or are identical fluid interfaces 37.

Consumables may be liquids, bags containing liquids, filters and the like.

This description of the interfaces may apply to any one, any combination of or all of the different cartridges 4 and/or receptacles 15 described herein. It can be that at least 50% of the cartridges 4 and/or of the types of cartridges 4 comprise standardized interfaces 6 with one or more of the described features.

The term “identical” always means identical in a function manner. Of course, tubes may lie around slightly differently for example.

A way for connecting tubes of different cartridges 4, receptacles 15 and/or functional elements is the tube welding described below. Other connection methods like sterile connectors may be used, too. In various embodiments, at least some, in some embodiments all, fluid interfaces 37 are used in defined positions with regard to the base structure 3 such that the base structure 3 can interact with the interfaces, in particular connect the tubes, in a standardized manner.

In various embodiments, a cartridge 4 and/or a receptacle 15 comprises a maximum of four inlets and outlets over all its liquid interfaces. In particular, each fluid interface 37 comprises exactly one potential fluid connection which can be used as an inlet or an outlet. For any one, any combination of or all described preconfigured cartridge 4 or preconfigured cartridges 4 at least one liquid interface may be used as an inlet and/or at least one liquid interface may be used as an outlet and/or at least one liquid interface may be used for two different connections, in particular to two different receptacles 15 and/or cartridges 4, consecutively. For any one, any combination of or all described preconfigured cartridge 4 or preconfigured cartridges 4 one liquid interface may be used as the only inlet and/or one liquid interface may be used as the only outlet and/or one liquid interface may be used as the product outlet and one liquid interface may be used as the only waste outlet and/or exactly two liquid interfaces may be used as inlets. A product outlet is an outlet for transferring the cell culture out of the cartridge 4.

In various embodiments, the fluid interface 37 comprises a part of a tube also placed inside the cartridge 4 and directly leading to a functional device 13 of the cartridge 4. In various embodiments, no change is made to the fluidic structure inside the cartridge 4 after its preconfiguration. The interface then only changes the cartridge fluidic structure 5 by connecting it to different elements, tubes or the like outside the cartridge 4.

According to one embodiment it is proposed, that the bioprocessing system 1 comprises receptacles 15, that the receptacles 15 are preconfigured with receptacle fluidic structures 16 for containing and providing and/or receiving liquids, the cell cultures and/or consumables that the bioprocessing system 1 uses in at least some of the operations, in various embodiments, that the receptacles 15 comprise fluid interfaces 37 identical to the fluid interfaces 37 of the cartridges 4.

The receptacles 15 may be the main way of transporting liquids within the bioprocessing system 1. In particular, the cell cultures may be transported from a receptacle 15 to a cartridge 4 prior to the operation or operations performed in the cartridge 4 and/or from a cartridge 4 to a receptacle 15 after the operation or operations performed in the cartridge 4. The receptacles 15 may be the only way the cell cultures are transported. However, the receptacles 15 may also be used, even only, for the transport of consumables being or containing liquids like waste, media, buffers or the like, for example. In various embodiments, the receptacles 15, in particular the receptacle liquid containers 56, may be filled at a media fill service station 55 of the bioprocessing system 1. The media fill service station 55 may comprise one or more tanks with liquids like media or buffers for many bioprocesses. This makes inputting these liquids into the bioprocess system 1 a lot easier as they do not have to be inputted in small doses for every time a liquid is needed.

In the following, the cartridges 4 usable in the bioprocessing system 1 will be described in detail. For any one, any combination of or all of the cartridges 4 that will now be described it may be the case that only operations of the respective named processing step (single step cartridge 4) or processing steps (shared cartridges 4) are performed inside the cartridge 4 or that only operations of one or two other processing step or processing steps are performed inside the cartridge 4. Further, any combination of the named cartridges 4 may be used by the bioprocessing system 1 for a single bioprocess and/or for different bioprocesses performed in parallel. Nevertheless, in various embodiments, one cartridge 4 is only used for one bioprocess.

According to one embodiment it is proposed, that the bioprocessing system 1 performs one or more or all operations of the processing step “enrichment” and/or of the processing step “wash” and/or of the processing step “separation” via the base structure 3 inside a preconfigured centrifuge cartridge 39 with a centrifuge chamber 40 as a functional device 13 inside the centrifuge cartridge 39.

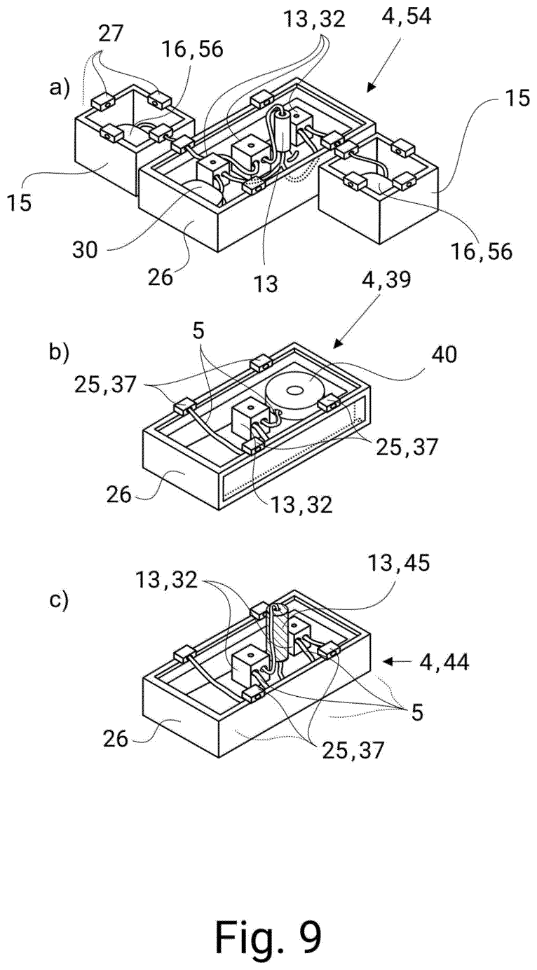

The centrifuge cartridge 39, exemplarily shown in FIG. 9, may be preconfigured by placing the centrifuge chamber 40 inside the centrifuge cartridge 39, in particular by placing the centrifuge chamber 40 in a predefined location. Some or all cartridges 4 may have such predefined locations. The predefined location may be a location dedicated to centrifuges or another functional device 13 or may be a location predefined for functional devices 13 of a certain size. For example, the predefined location may comprise connection elements, like screws or screw openings, clip elements or the like. The cartridges 4 may comprise multiple overlapping predefined locations for different sizes of functional elements, whereby, in some embodiments, the differently sized functional elements utilize different combinations of the connection elements depending on their size and/or location. For example, the floor of the cartridge 4 may be designed as a plug-in board.

The centrifuge chamber 40 may be a fluidized bed centrifuge chamber 40.

As generally denoted by the use of “and/or”, it is to be understood that different cartridges 4 for differently performed processing steps of the same type may be used in a single bioprocessing system 1 by differently preconfigured cartridges 4 for example.

Here and in the following, various configurations of inlets and outlets are described. Every inlet and/or outlet is part of a fluid interface 37. Of course, the definition of the inlets and outlets is valid for the respective use of the preconfigured cartridge 4 and may change with a different configuration.

In various embodiments, the centrifuge cartridge 39 comprises exactly one dedicated inlet for the cell culture and/or exactly one or exactly two dedicated inlets for a buffer. Alternatively, the centrifuge cartridge 39 may comprise exactly one inlet, in particular for the cell culture and the buffer. The centrifuge cartridge 39 may comprise exactly one waste outlet and/or exactly one product outlet. The inlets and/or outlets may be directly connected to the centrifuge chamber 40.

The centrifuge cartridge 39 may further comprise one or more, in particular single-use, flow sensors and/or pumps.

The cartridge interface 8 of the centrifuge cartridge 39 may comprise and use a connection for mechanical and/or electrical energy and/or for control signals for the centrifuge chamber 40 and/or from the flow sensor and/or for the pumps. A centrifuge control chip receiving the sensor signals from the flow sensor and controlling the centrifuge may also be placed inside the centrifuge cartridge 39 and may be powered via the cartridge interface 8.

According to one embodiment it is proposed, that the bioprocessing system 1 performs one or more or all operations of the processing step “enrichment” and/or of the processing step “separation” via the base structure 3 inside a preconfigured acoustics cartridge 41 with at least one acoustic wave generator, in particular piezoelectric element, as a functional device 13 inside the acoustics cartridge 41.

The acoustics cartridge 41, exemplarily shown as an exemplary cartridge 4 with a functional interface schematically in FIG. 10, may be preconfigured by placing the acoustic wave generator inside the acoustics cartridge 41, in particular by placing the acoustic wave generator in a predefined location. The acoustic wave generator may comprise one or two transducers, in particular interdigital transducers. The acoustic wave generator may further comprise an acoustic reflector. The acoustics cartridge 41 may be identical to the centrifuge cartridge 39 prior to being preconfigured. The acoustic wave generator and eventually the reflector may be contained in a modular small cartridge 42 that can be placed inside the acoustics cartridge 41, in some embodiments be clipped into the acoustics cartridge 41. The acoustic wave generator may contain tubes or the like and may be part of the cartridge fluidic structure 5. Alternatively, tubes or the like may be inserted into the small cartridge 42 at preconfiguration.

FIG. 9 also shows how the centrifuge cartridge 39 may have a different functional interface than the acoustics cartridge 41 even though the functional interface of the centrifuge cartridge 39 is not used there.

Acoustic separation of cells works by applying acoustic waves to the cartridge fluidic structure 5 and creating a standing wave. The cell culture is flooded together with a sheath flow buffer through the fluidic structure with the standing wave and the cells separate based on their size into different channels of the fluidic structure.

In various embodiments, the acoustics cartridge 41 comprises exactly one dedicated inlet for the cell culture and/or exactly one dedicated inlet for a buffer. A continuous stream of sheath flow may be provided via the buffer inlet during the enrichment or separation, in particular from a receptacle 15. The acoustics cartridge 41 may comprise exactly one waste outlet and/or exactly one product outlet. The inlets and/or outlets may be directly connected to a part of the cartridge fluidic structure 5 to which the acoustic waves of the acoustic wave generator are applied.

The acoustics cartridge 41 may further comprise one or more, in particular single-use, flow sensors and/or pumps and/or a bubble sensor and/or a temperature sensor for the acoustic wave generator and/or for the cell culture. The acoustics cartridge 41 may comprise a cooling arrangement for the acoustic wave generator, which may be active or passive. The cooling arrangement may be powered via the standardized interfaces 6 if it is an active cooling arrangement. The cooling arrangement may comprise one or more cooling elements, for example cooling ribs and/or a ventilator. The cooling arrangement may be part of the acoustics cartridge 41 prior to its preconfiguration or added to the acoustics cartridge 41 as part of its preconfiguration.

The cartridge interface 8 of the acoustics cartridge 41 may comprise and use a connection for electrical energy and/or for control signals for the pumps and/or the acoustic wave generator and/or from the flow sensor and/or from the temperature sensor. An acoustics control chip receiving the sensor signals from the flow sensor and/or the bubble sensor and/or the temperature sensor and controlling the acoustic wave generator and/or the pumps may also be placed inside the centrifuge cartridge 39 and may be powered via the cartridge interface 8. In case the temperature of the acoustic wave generator or the cell culture reaches a predefined threshold, the control chip may stop the acoustic wave generator.

According to one embodiment it is proposed, that the bioprocessing system 1 performs one or more or all operations of the processing step “enrichment” and/or of the processing step “separation” via the base structure 3 inside a preconfigured acoustics cartridge 41 with at least one acoustic wave generator as a functional device 13 outside the acoustics cartridge 41.

Placing the acoustic wave generator outside the cartridge 4 allows for easy re-use of the acoustic wave generator. In various embodiments, the acoustic wave generator can apply the acoustic waves to the cartridge fluidic structure 5, in particular flow channels of the cartridge fluidic structure 5, of the acoustics cartridge 41 or comprises automatically changeable and/or sterilizable tubes or the like forming the flow channels and connectable to the cartridge fluidic structure 5 of the acoustics cartridge 41.

It may be the case that the acoustics cartridge 41 comprises a functional interface to the acoustic wave generator, that the acoustics cartridge 41 comprises a fluidic structure to which the acoustic waves are applied via the functional interface from one or more sides, in various embodiments, that the acoustics cartridge 41 comprises a reflector for the acoustic waves. A part of the cartridge fluidic structure 5 may comprise the tubes or the like for application of the acoustic waves in a defined manner with a defined placement, in particular at the edge of the acoustics cartridge 41 as part of the functional interface. The acoustic wave generator may be docked onto the functional interface. The functional interface may comprise placement elements 43 ensuring the precise fit between the cartridge 4 and the acoustic wave generator. A reflector may be part of the acoustics cartridge 41.

The functional device 13 can be placed in a standard container 36 with a standard connection to the cartridge 4 for different technologies like acoustic waves, optical energy application, electromagnetic fields, etc.

A cartridge 4 can have a standard slot for adapted small cartridges 42 comprising the fluidic structure adapted for the energy application, possibly a reflector or the like and placeable in a standard position of the cartridge 4 via for example a clip mechanism (FIG. 10a).

Any or all of the flow channels within the acoustic cartridge 41 can, in use, have a monitoring system attached. This monitoring system can be used to regulate the flow of liquid through the channels, for example, by changing pumping rates. It can also be used to detect faults within the flow channels for example blockages, air bubbles or manufacturing issues such as narrow channels or inadequate coupling of the acoustics. Exemplarily, a monitoring system would be an optical microscope focussed on a flow channel able to detect the presence and sizes of cells passing through the channel for example under stroboscopic light or using low exposures.

The monitoring system may be part of the acoustics cartridge 41. Alternatively, the functional interface between the acoustics cartridge 41 and the acoustic wave generator may also be used for the monitoring system. In that case, the monitoring system may be part of the base structure and/or of a container 36 comprising the acoustics wave generator.

The same concept can be applied to other operations of the same or other processing steps, in particular other operations of separation and/or selection processing steps, to detect and/or modulate the correct functioning of those operations. Therefore, one or more standard containers 36 may generally comprise a monitoring system.

According to one embodiment it is proposed, that the bioprocessing system 1 performs one or more or all operations of the processing step “enrichment” and/or of the processing step “separation” via the base structure 3 inside a preconfigured DLD cartridge 4 with a deterministic lateral displacement chamber as a functional device 13 inside the DLD cartridge 4.

The DLD cartridge 4 may be preconfigured by placing the deterministic lateral displacement chamber inside the DLD cartridge 4, in particular by placing the deterministic lateral displacement chamber in a predefined location.

In various embodiments, the DLD cartridge 4 comprises exactly one dedicated inlet for the cell culture and/or exactly one dedicated inlet for a buffer. Alternatively, the DLD cartridge 4 may comprise exactly one inlet, in particular for the cell culture and the buffer. The DLD cartridge 4 may comprise exactly one waste outlet and/or exactly one product outlet. The inlets and/or outlets may be directly connected to the deterministic lateral displacement chamber.

The DLD cartridge 4 may further comprise one or more, in particular single-use, flow sensors. The DLD cartridge 4 may comprise a valve for starting and stopping a buffer flow and/or for starting and stopping a flow of the cell culture. Additionally or alternatively, the DLD cartridge 4 may comprise at least one pump to control the flow of buffer and/or cell culture.

In general, other means for liquid transport besides pumps may also be used to guide a liquid within the tubes.

The cartridge interface 8 of the DLD cartridge 4 may comprise and use a pneumatic connection to drive liquids and/or actuate the valve and/or for control signals for the valve and/or from the flow sensor. It is to be understood that the exemplary descriptions of the cartridges 4 are not limiting, for example, the DLD cartridge 4 could also comprise an electrically actuated valve and therefore an electrical connection for actuating the valve.

According to one embodiment it is proposed, that the bioprocessing system 1 performs one or more or all operations of the processing step “enrichment” and/or of the processing step “separation” via the base structure 3 inside a preconfigured electric and/or magnetic separation cartridge 4 with at least one sorting element, in particular an electric and/or magnetic field generation element, as a functional device 13 inside the magnetic separation cartridge 4.

In various embodiments, the electric and/or magnetic separation cartridge 4 is a flow cytometry cartridge 4 and comprises at least one laser and a detection optic and at least one, in some embodiments at least eight or at least sixteen, sorting elements, in particular charged plates, as functional devices 13 inside the flow cytometry cartridge 4.

Alternatively, the electric and/or magnetic separation cartridge 4 may be a negative magnetic immunoadherence cartridge 4 and comprise at least one magnetic field generation element inside the cartridge 4.

A cell marking for the electric and/or magnetic separation may be performed in an incubator service station 38 of the bioprocess system 1 or in the electric and/or magnetic separation cartridge 4. The cell marking may be performed in the electric and/or magnetic separation cartridge 4 and afterwards, the cell culture may be transferred to an incubator location 52. The transfer may be performed by transferring the electric and/or magnetic separation cartridge 4 or by transferring the cell culture to a receptacle 15 and returning it later.

In various embodiments, the electric and/or magnetic separation cartridge 4 comprises exactly one dedicated inlet for the cell culture and/or exactly one dedicated inlet for a buffer. A continuous stream of sheath flow may be provided via the buffer inlet during the enrichment or separation, in particular from a receptacle 15. The electric and/or magnetic separation cartridge 4 may comprise exactly one waste outlet and/or exactly one product outlet. The inlets and/or outlets may be directly connected to a part of the cartridge fluidic structure 5 to which the electric and/or magnetic separation energy is applied.

The cartridge interface 8 of the electric and/or magnetic separation cartridge 4 may comprise and use a connection for mechanical and/or electrical energy and/or for control signals for the laser and/or the detection optic and/or the sorting element and/or the magnetic field generation element. An electric and/or magnetic separation control chip may also be placed inside the centrifuge cartridge 39 and may be powered via the cartridge interface 8.

According to one embodiment it is proposed, that the bioprocessing system 1 performs one or more or all operations of the processing step “enrichment” and/or of the processing step “separation” via the base structure 3 inside a preconfigured electric and/or magnetic separation cartridge 4 with at least one sorting element, in particular an electric and/or magnetic field generation element, as a functional device 13 outside the cartridge 4, in various embodiments, that the electric and/or magnetic separation cartridge 4 is a flow cytometry cartridge 4 and the bioprocessing system 1, in particular the base structure 3, comprises at least one laser and a detection optic and at least one sorting element, in particular charged plates, as functional devices 13 outside the flow cytometry cartridge 4, or, that the electric and/or magnetic separation cartridge 4 is a negative magnetic immunoadherence cartridge 4 and the bioprocessing system 1, in particular the base structure 3, comprises at least one magnetic field generation element outside the electric and/or magnetic separation cartridge 4.

Placing the sorting element outside the cartridge 4 allows for easy re-use of the sorting element. In various embodiments, the sorting element can apply the electric and/or magnetic field to the cartridge fluidic structure 5 of the electric and/or magnetic separation cartridge 4 or comprises automatically changeable and/or sterilizable tubes or the like connectable to the cartridge fluidic structure 5 of the electric and/or magnetic separation cartridge 4.

It may be the case that the electric and/or magnetic separation cartridge 4 comprises a functional interface to the sorting element. A part of the cartridge fluidic structure 5 may comprise the tubes or the like for application of the electric and/or magnetic field in a defined manner with a defined placement, in particular at the edge of the electric and/or magnetic separation cartridge 4 as part of the functional interface. The sorting element may be docked onto the functional interface. The functional interface may comprise placement elements 43 ensuring the precise fit between the cartridge 4 and the sorting element. The laser may also be located outside the cartridge 4.

The cartridge 4 can have a standard slot for adapted small cartridges 42 comprising the fluidic structure adapted for the electric and/or magnetic field application.

According to one embodiment it is proposed, that the bioprocessing system 1 performs one or more or all operations of the processing step “enrichment” and/or the processing step “wash” and/or of the processing step “separation” via the base structure 3 inside a preconfigured filter cartridge 44 with at least one filter 45 as a functional device 13 inside the cartridge 4. FIG. 9 also shows a filter cartridge 44. The shown cartridges 4 serve as examples for the other possible cartridges 4.

In various embodiments, the filter cartridge 44 comprises exactly one dedicated inlet for the cell culture and/or exactly one dedicated inlet for a buffer. The filter cartridge 44 may comprise exactly one waste outlet and/or exactly one product outlet. The inlets and/or outlets may be directly connected to the filter 45.

The filter cartridge 44 may further comprise one or more, in particular single-use, flow sensors and/or a pump, in particular for generating a transmembrane pressure for the filter 45.

The cartridge interface 8 of the filter cartridge 44 may comprise and use a connection for electrical energy and/or for control signals for the pump and/or from the flow sensor. A filter 45 control chip receiving the sensor signals from the flow sensor and controlling the pump may also be placed inside the filter cartridge 44 and may be powered via the cartridge interface 8.

In various embodiments, the filter 45 is preconfigured as part of the cartridge fluidic structure 5.

According to one embodiment it is proposed, that the bioprocessing system 1 automatically connects the centrifuge cartridge 39 and/or the acoustics cartridge 41 and/or the DLD cartridge 4 and/or the electric and/or magnetic separation cartridge 4 and/or the filter cartridge 44, in particular via one of the fluid interfaces 37, to a cartridge 4 or receptacle 15 containing the cell culture prior to the processing step and/or to a receptacle 15 containing buffer as a consumable and/or to a receptacle 15 for receiving a washed or separated or enriched portion of the cell culture after the processing step and/or to a receptacle 15 for receiving a waste portion of the cell culture and/or of the consumable, in various embodiments, that the cell culture and the buffer are provided via the same fluid interface 37.

The tube connection system 23, particularly the tube welding system 24 may be used for any of these connections.

In various embodiments, the centrifuge chamber 40 and/or the deterministic lateral displacement chamber and/or the filter 45 are single-use components with regard to the bioprocessing system 1, meaning that they may be sterilized but have to leave the bioprocessing system 1 for that. A bioprocess may comprise more than one of the processing steps “enrichment”, “separation” and “wash”, in particular a bioprocess may comprise at least two processing steps “wash”.

It may be the case that the bioprocessing system 1 reuses one centrifuge cartridge 39 and/or DLD cartridge 4 for multiple processing steps of one and the same bioprocess, with or without a processing step “wash”and possibly disinfection step in between.

According to one embodiment it is proposed, that the bioprocessing system 1 performs one or more or all operations of the processing step “selection” via the base structure 3 inside a preconfigured magnetic selection cartridge 54 with at least one magnetic field generation element as a functional device 13 inside or outside the cartridge 4, in various embodiments, that the magnetic selection cartridge 54 is the electric and/or magnetic separation cartridge 4 or that the electric and/or magnetic separation cartridge 4 and the magnetic selection cartridge 54 are combined into a shared cartridge 4, in some embodiments, that the electric and/or magnetic separation cartridge 4 is a magnetic separation cartridge 4 that shares one or more functional devices 13 with the magnetic selection cartridge 54 inside the shared cartridge 4.

The shared functional device 13 may act on the same part of the cartridge fluidic structure 5 or different parts of the cartridge fluidic structure 5 for the different processing steps. Both processing steps may be separate processing steps, the product may even leave the cartridge 4 in between, for example for coating or incubation.

All explanations given with regard to the functional elements of the electric and/or magnetic separation cartridge 4 may apply here, too.

The magnetic selection can be performed on cell cultures incubated with magnetic beads coated with antibodies, in particular antibodies targeting CD62L and/or CD4 and/or CD8 and/or CD56 and/or CD3.

Instead of or additionally to selecting the target cells via magnet selection, unwanted cells may be selected via magnetic selection.

In various embodiments, the magnetic selection cartridge 54 comprises exactly one dedicated inlet for the cell culture, in particular a cell culture incubated with magnetic beads, and/or exactly one dedicated inlet for a magnetic bead comprising liquid. The magnetic selection cartridge 54 may comprise exactly one waste outlet and/or exactly one product outlet. The inlets and/or outlets may be directly connected to the functional device 13.

The cartridge interface 8 of the magnetic selection cartridge 54 may comprise and use a connection for electrical energy and/or for control signals for the magnetic field generation element. A magnetic field generation element control chip may also be placed inside the magnetic selection cartridge 54 and may be powered via the cartridge interface 8.

A magnet selection can be performed by mixing the cell culture with magnetic beads, for example in the magnetic selection cartridge 54 or in a mixing cartridge or at the incubator service station 38, then incubating the cell culture, then, if necessary, transferring the cell culture into the magnetic selection cartridge 54, then flowing the cell culture through a column of the magnetic selection cartridge 54, then removing or deactivating the magnetic field generation element, in particular by removing the container containing the magnetic field generation element or disabling an electromagnet, then flowing a buffer, in particular through an inlet of the magnetic selection cartridge 54, through the column and capturing the remainder as the cell culture, in particular behind an outlet of the magnet selection cartridge 54.

The waste may be transferred from the magnetic selection cartridge 54 or any other yet to be described selection cartridge 4 after the selection to a storage position, in particular inside a receptacle 15. The same may be true for the cartridges 4 in which enrichment and/or wash and/or separation are performed. This way, the stored receptacles may still be used, for example to repeat the step of “selection”.

If a criterion relating to a number or viability or the like of target cells is not met, the waste may be submitted to another processing step, in particular selection step, to gain more target cells. Thereby, the bioprocess may be flexibly changed to include more steps, the waste becoming the cell culture for gaining more target cells. A further combination step of both portions of target cells may be done automatically. Alternatively, the waste may be submitted to the same processing step to repeat that step.

According to one embodiment it is proposed, that the bioprocessing system 1 performs one or more or all operations of the processing step “selection” via the base structure 3 inside a preconfigured buoyancy selection cartridge 4 with at least one centrifuge as a functional device 13 inside or outside the cartridge 4, in various embodiments, that the buoyancy selection cartridge 4 is the centrifuge cartridge 39 or that the centrifuge cartridge 39 and the buoyancy selection cartridge 4 are combined into a shared cartridge 4, in some embodiments, that the centrifuge cartridge 39 shares the centrifuge chamber 40 with the buoyancy selection cartridge 4 inside the shared cartridge 4.

The buoyancy selection can be performed on cell cultures incubated with gas-filled lipid-shell microbubbles coated with antibodies, in particular antibodies targeting CD28 and/or CD3.

In various embodiments, the buoyancy selection cartridge 4 comprises exactly one dedicated inlet for the cell culture and/or exactly one dedicated inlet for a microbubbles comprising liquid. The buoyancy selection cartridge 4 may comprise exactly one waste outlet and/or exactly one product outlet. The inlets and/or outlets may be directly connected to the functional device 13.

The cartridge interface 8 of the buoyancy selection cartridge 4 may comprise and use a connection for electrical energy and/or for control signals for the centrifuge. A centrifuge control chip may also be placed inside the buoyancy selection cartridge 4 and may be powered via the cartridge interface 8.

According to various embodiments it is proposed, that the bioprocessing system 1 performs one or more or all operations of the processing step “activation” via the base structure 3 inside a preconfigured activation cartridge 4, in some embodiments with at least one pump as a functional device 13 inside or outside the cartridge 4.

The activation may comprise addition of liquid, incubation and washing. Those steps may be performed in one cartridge 4 or separate cartridges 4. It may be the case that the activation is not performed in a dedicated activation cartridge 4.

The activation may comprise adding soluble antibodies or paramagnetic beads coated with antibodies or an antibody containing nanomatrix to the cell culture. The magnetic beads or the nanomatrix may be removed by washing afterwards.

The activation cartridge 4 may comprise a cell count sensor and/or a mixing volume and/or a pump. The cartridge interface 8 of the activation cartridge 4 may comprise and use a connection for electrical energy and/or for control signals for the pump and/or from the cell count sensor.

The activation may be performed at an incubator service station 38. The liquid for activation may be added to the activation cartridge 4 and/or at the incubator service station 38 and/or at a media fill service station 55. The incubation may be performed inside or outside the activation cartridge 4.

According to one embodiment it is proposed, that the bioprocessing system 1 performs one or more or all operations of the processing step “activation” via the base structure 3 inside the centrifuge cartridge 39 and/or the acoustics cartridge 41 and/or the DLD cartridge 4 and/or the electric and/or magnetic separation cartridge 4 and/or the filter cartridge 44 as a shared cartridge 4.

The functional device 13 may be reused for at least an operation of the processing step “enrichment” or the processing step “wash” or the processing step “separation” and at least an operation of the processing step “selection” and/or an operation of the processing step “activation”. To make it clear, one step of enrichment, wash or separation may be combined with one step of selection or activation. Further steps of the other processing steps may be added.

It may be the case, that this shared cartridge 4 comprises a pump and/or an acoustic wave generator and/or a magnetic field generation element and/or a centrifuge as a functional device 13 or is connected to the functional device 13 via a functional interface.

In various embodiments, flow cytometry or a microscope can be used to monitor activation. Activation results in cells changing in diameter from around 6 to 11 microns and is therefore detectable with transmission light microscopy of unstained cells. The microscope may be part of the respective cartridge 4 or a container connected via a functional interface.

According to various embodiments it is proposed, that the bioprocessing system 1 performs one or more or all operations of the processing step “genetic modification” via the base structure 3 inside a preconfigured genetic modification cartridge 4, in some embodiments, that the activation cartridge 4 and the genetic modification cartridge 4 are one shared cartridge 4.

This shared cartridge 4 may comprise a pump and/or a centrifuge as functional device 13. The activation and the genetic modification may together be performed in a cartridge 4 shared with other processing steps as described for the activation. The activation and/or the genetic modification may be performed by spinoculation inside a cartridge 4, in particular the shared cartridge 4, comprising a centrifuge.

According to various embodiments it is proposed, that the bioprocessing system 1 performs one or more or all operations of the processing step “genetic modification” via the base structure 3 inside the centrifuge cartridge 39 and/or the acoustics cartridge 41 and/or the DLD cartridge 4 and/or the electric and/or magnetic separation cartridge 4 and/or the filter cartridge 44 as a shared cartridge 4, in some embodiments, that the functional device 13 is reused for at least an operation of the processing step “enrichment” or the processing step “wash” or the processing step “separation” and at least an operation of the processing step “selection”and/or an operation of the processing step “genetic modification”.

Further it may be the case, that the bioprocessing system 1 performs the genetic modification by viral transduction or electroporation or nanoparticle-based delivery. A viral transduction enhancing reagent may be added through an inlet of the genetic modification cartridge 4, in particular from a receptacle 15. Alternatively, the viral transduction enhancing reagent may be placed inside a cartridge liquid container 30 during preconfiguration. The viral transduction enhancing reagent may comprise RetroNectin or cationic polymers, e.g., Polybrene, or cationic lipids.

The electroporation comprises mixing the cell cultures such as with DNA or mRNA in an electroporation buffer. The genetic modification cartridge 4 may comprise an inlet for the DNA or mRNA and/or an inlet for the buffer. Further, the genetic modification cartridge 4 may comprise one or two inlets for a high-conductivity sheath medium.

The genetic modification cartridge 4 may comprise an electroporation cuvette and/or an electric field generation element inside the cartridge 4. A reuse of the electrical field generation element, in particular charged plates, of the electric separation cartridge, in particular in a shared cartridge 4 or of the electrical field generation element as part of the base structure 3, is possible.

In various embodiments, the bioprocessing system 1 performs one or more or all operations of the processing step “expansion” outside a cartridge 4 and/or at an expansion location and/or inside a receptacle 15. In various embodiments, at least one operation of the processing step “expansion”is performed at an incubator service station 38.

According to one embodiment it is proposed, that the bioprocessing system 1 performs one or more or all operations of the processing steps “formulation” and “fill” via the base structure 3 inside a preconfigured formulation cartridge 4.

The formulation cartridge 4 may comprise an inlet for the cell culture and/or an inlet for a formulation buffer. It may comprise an outlet for the cell culture or no outlet for the cell culture, in particular if it is the last processing step. The formulation cartridge 4 may contain the final product packaging and the cell culture may be added inside the formulation cartridge 4.

According to various embodiments it is proposed, that the bioprocessing system 1 performs one or more or all operations of the processing steps “formulation” and “fill” via the base structure 3 inside the centrifuge cartridge 39 and/or the acoustics cartridge 41 and/or the DLD cartridge 4 and/or the electric and/or magnetic separation cartridge 4 and/or the filter cartridge 44 and/or the activation cartridge 4 and/or the genetic modification cartridge 4 as a shared cartridge 4, in some embodiments, that the functional device 13 is reused for at least an operation of the processing step “enrichment” or the processing step “wash” or the processing step “separation” and at least an operation of the processing step “selection” and/or an operation of the processing steps “formulation” and “fill”.

The bioprocessing system 1 may have dedicated stations for processing steps with functional devices 13 outside the cartridges 4 and general stations for processing steps with functional devices 13 inside the cartridges 4. At the general stations different operations of different processing steps may be performed by the bioprocessing system 1 at different times with different cartridges 4.

Turning back to the general explanations of the proposed method, it may be the case that in a not preconfigured state a cartridge 4 is not yet usable for any of the processing steps or operations by the bioprocessing system 1.

To clarify some embodiments, the first processing steps of a bioprocess for manufacturing genetically modified CAR-T cells from a liquid immune cell culture will be described in the following.

As may best be seen from FIG. 4, a receptacle 15 containing an initial liquid immune cell culture is introduced into the integrated bioprocessing system 1 by an operator at an input-output location 46. From the input-output location 46, the receptacle 15 is transferred to a transfer location 14 of a unit operation station 2 by the global transport mechanism 17. In the example given, the unit operation station 2 is being preconfigured to the enrichment processing step by performing the standard routine on the respective cartridge 4, that has been preconfigured accordingly. In detail, the preconfigured cartridge 4 is being transported from the cartridge storage unit 9 to the cartridge drive unit 11.

In various embodiments, the unit operation of the enrichment processing step at least comprises a counter-flow centrifugation unit operation step. However, as mentioned above, instead of counter-flow centrifugation, also acoustic separation may be used. For counter-flow centrifugation, the cartridge fluidic structure 5 of the respective cartridge 4 comprises a counter-flow centrifuge chamber 40 to receive a liquid to be centrifuged. Accordingly and as mentioned above, the counter-flow centrifuge chamber 40 can be made of a single-use material.

Before, after or during the transport of the receptacle 15 to the transfer location 14 of the unit operation station 2, the cartridge 4 of the unit operation station 2, that comprises a cartridge fluidic structure 5 that has been preconfigured to the enrichment processing step, is brought into operative coupling with the cartridge drive unit 11. In detail, the counter-flow centrifuge chamber 40 of the cartridge fluidic structure 5 is brought into operative coupling with a standardized mechanical interface of the cartridge drive structure 33 as part of the operative coupling. This mechanical interface may comprise a rotor that is used to rotate the counter-flow centrifuge channel. As mentioned above, the rotor can be designed to be used multiple times.

After the receptacle 15 containing the liquid immune cell culture has been transported to the transfer location 14 of the unit operation station 2 and the cartridge 4 has been transported to the drive location 10, the receptacle transfer tube 22 and one of the cartridge transfer tubes 21 are connected by welding in the closed connection process (FIG. 6a).

Next, the liquid immune cell culture is transferred via the welded connection from the receptacle 15 to the counter-flow centrifuge element of the cartridge 4 using a fluid interface 37 and a peristaltic pump 32 to transfer the liquid immune cell culture to the centrifuge chamber 40 (FIG. 3). Again, the respective interface for engaging the peristaltic pump 32 is provided by the base structure 3, in particular by the base structure interface 7.

Next, counter-flow centrifugation is carried out on the liquid immune cell culture by the counter-flow centrifuge of the cartridge 4. For this, the counter-flow centrifuge chamber 40 is fluidically connected to a cartridge liquid container 30 that may receive the waste liquid from the counter-flow centrifugation unit operation step. Alternatively, the counter-flow centrifuge chamber 40 is fluidically connected to a receptacle 15 provided in a transfer location 14 with the receptacle 15 comprising a receptacle fluidic structure 16 not yet containing a liquid. The receptacle 15 may be transported to the transfer location 14 by the global transport mechanism 17 and may be provided from a central supply storage 47.

Here it should be noted, that at least part of the liquids required at a respective unit operation station 2 for performing a unit operation may also be supplied from the media fill service station 38 after the respective liquid has been transferred into a receptacle 15 at a media fill location 48 of the media fill service station 38 as described above. In this case, the receptacle 15 is transported to a transfer location 14 of the particular unit operation station 2 by the global transport mechanism 17 and the connection between the cartridge 4 and the receptacle 15 is established in a connection process as described above.

Optionally, a washing unit operation step may be carried out after the counter-flow centrifugation unit operation step (not depicted). For this, the cartridge 4 may comprise at least one cartridge liquid container 30 with a washing liquid. The washing liquid is transferred from the cartridge liquid container 30 to the counter-flow centrifuge element. Alternatively, the washing liquid may be provided from the media fill service station 38. In the latter case, the washing liquid is transferred from a media storage container 36 to a receptacle 15 liquid container 36 as described above. Afterwards, the receptacle 15 is transported by the global transport mechanism 17 to a transfer location 14 of the unit operation station 2 and connected to the cartridge 4 in the closed connection process also described above.

Again, the waste liquid from the washing unit operation step is transferred to a cartridge liquid container 30. Alternatively, the waste liquid is transferred to a receptacle liquid container 56 of a receptacle 15 provided in a transfer location 14. In various embodiments, the same liquid container that was also used to receive the waste liquid from the centrifugation of the liquid immune cell culture is used to receive the waste liquid from the washing unit operation step. However, in an alternative embodiment, the cartridge fluidic structure 5 may comprise different cartridge liquid containers 30 to receive the waste liquid and/or different receptacle liquid containers 56 may be used. After transfer of the liquid waste of the washing unit operation step, the now purified liquid immune cell culture may be eluted from the counter-flow centrifuge element and transferred to a second receptacle 15 that is provided in a second transfer location 14 of the unit operation station 2 (FIG. 3). Again, a connection between the receptacle transfer tube 22 of the second receptacle 15 and a cartridge transfer tube 21 of the cartridge 4 is established in a closed connection process. For elution, the cartridge 4 may comprise another cartridge liquid container 30 that comprises an elution liquid like culture medium or the like (not depicted). The purified T cells are eluted from the counter-flow centrifuge to a receptacle liquid container 56 of the second receptacle 15.

After all unit operation steps of the unit operation of the enrichment processing step have been performed, the welded connections between the receptacle transfer tubes 22 and the respective cartridge transfer tubes 21 of the cartridge 4 are disconnected in the disconnecting process. Afterwards, the cartridge 4 is transferred from the cartridge drive unit 11 to a cartridge waste storage unit 49.

After disconnection, the first receptacle 15, now comprising an empty receptacle liquid container 56, may be transported by the global transport mechanism 17 to a central waste storage 50 to be discarded. The second receptacle 15, now containing the partly processed liquid immune cell culture, is transferred by the global transport mechanism 17 to the unit operation station 2, which is preconfigured for the selection processing step.

During the selection processing step, a certain subtype of the liquid immune cells within the liquid immune cell culture is enriched. In various embodiments, a subtype of T cells is enriched. In various embodiments, a subtype of T cells expressing the antigen CD4, CD8 or CD62L is enriched. In a proposed embodiment, the unit operation of the selection processing step comprises at least a magnetic separation unit operation step.

For the selection processing step, the receptacle 15 containing the liquid immune cell culture after the enrichment processing step, is now transported to a transfer location 14 of a particular unit operation station 2, which is preconfigured for the selection processing step by preconfiguration of one of its cartridges 4. As explained for the enrichment processing step, the cartridge 4 for the unit operation of the selection processing step, which is stored in the cartridge storage unit 9 of one of the unit operation stations 2, has been moved into the drive location 10 of this particular unit operation station 2.

After the connection process for establishing a fluidic connection between the receptacle 15 and the cartridge 4, from a cartridge liquid container 30, a liquid comprising magnetic beads that have been coated with an antibody directed against an antigen on the T cell surface, is transferred into the receptacle 15 containing the liquid immune cell culture in a liquid addition unit operation step. Again, the liquid connection is established by the welding system 24 connecting the respective transfer tubes in a closed manner.

As an example, magnetic beads covered with an antibody directed against CD4 are added to the liquid immune cell culture and cells within the liquid immune cell culture comprising the CD4 surface antigen now attach to the magnetic beads.

In a next step, the receptacle 15 comprising the liquid immune cell culture and the magnetic beads may be transported to an incubator service station 38 that is located in a second plane 51. The incubator service station 38 comprises at least one incubator location 52 to receive the receptacle 15. In an embodiment and as depicted in FIG. 4, the incubator service station 38 may be designed as a drawer system. Alternatively, the incubator service station 38 may also be designed as a shelf system. However, also other configurations are imaginable. Again, the global transport mechanism 17 is used for the transport and an elevator system 53 may be used to transfer the receptacle 15 from the first plane 19 to the second plane 51 (FIG. 4). It should be noted, that while the incubator service station 38, including the at least one incubator location 52, is located in the second plane 51 in the example described here, it is also well possible that at least one incubator service station 38 may be located in the first plane 19.

At the incubator service station 38, the receptacle 15 is subjected to defined process conditions for a predefined amount of time in an incubation step. The incubation step is carried out to enable binding of the target immune cells (in this case CD4+ T cells) to the magnetic particles via the antigen-antibody bond. Hence, T cells comprising a certain antigen corresponding to the antibody coated to the magnetic particles, are bound to the magnetic particles.

After the incubation step, the receptacle 15 is being transported from the incubator service station 38 back to the unit operation station 2, that is preconfigured for the selection processing step.

Next, magnetic selection may be carried out on the liquid immune cell culture by the electromagnet or magnet of the magnetic selection cartridge 54. As described above, the addition of the magnetic beads to the liquid immune cell culture may have been performed in the magnetic selection cartridge 54 or it may have been performed at another cartridge 4 or at the incubator service station 38 of the bioprocessing system 1.

The liquid comprising the CD4+ cells attached to the magnetic beads as well as other cells not attached to the magnetic beads are introduced into the flow channel that is surrounded by the electromagnet as the functional device 13 via a first inlet. While the cells flow through the channel, the electromagnet is switched on to keep the magnetic beads and the cells attached to the magnetic beads in the part of the flow channel that is influenced by the electromagnet. Accordingly, cells not attached to magnetic beads leave the flow channel via a first outlet. The first outlet is fluidically connected to a cartridge liquid container 30 that may receive the waste liquid from the magnetic selection operation. Alternatively, to receive the waste liquid, the first outlet of the channel is fluidically connected to a receptacle 15 provided in a transfer location 14 with the receptacle 15 comprising a receptacle fluidic structure 16 not yet containing a liquid. The receptacle 15 may be transported to the transfer location 14 by the global transport mechanism 17 and may be provided from a central supply storage 47. Additionally, a second outlet of the magnetic channel is fluidically connected to a receptacle 15 provided in a second transfer location 14 with the receptacle 15 comprising a receptacle fluidic structure 16 not yet containing a liquid. After the immune cell culture has been passed through the channel, the magnet is switched off. Now, all previously bound magnetic beads, are released and may leave the area of the electromagnet. In various embodiments, the magnetic beads are directed into the second receptacle 15. After the target cells (CD4+ cells) have been collected in the second receptacle 15, the cells may be separated from the magnetic beads and subjected to the activation processing step.

Here it should be noted, that at least part of the liquids required at a respective unit operation station 2 for performing a unit operation may also be supplied from the media fill service station 38 after the respective liquid has been transferred into a receptacle 15 at a media fill location 48 of the media fill service station 38. In this case, the receptacle 15 is transported to a transfer location 14 of the particular unit operation station 2 by the global transport mechanism 17 and the connection between the cartridge 4 and the receptacle 15 is established in a connection process as described above.

All connection steps between the cartridge 4 and the receptacles 15 may be performed in a closed connection process, as described above.

According to one embodiment it is proposed, that the cartridges 4 and/or the receptacles 15 and/or the containers 36 are moved by the bioprocessing system 1 automatically, such as by the same transport mechanism, in particular a robotic manipulator.

According to various embodiments it is proposed, that the shared cartridge 4 has a wall separating the cartridge 4 into compartments for the different processing steps, in some embodiments, that the functional device 13 is part of the wall, and/or, that the cell culture is transferred through a fluidic structure passing through the wall.

As has already been explained, it may be the case that an electronic process control 28 of the bioprocessing system 1 may repeat a processing step or an operation if a criterion for the cell culture is not met during or after the processing step, thereby flexibly reconfiguring the bioprocess, and/or, that variations of processing steps may be performed via the same standardized base structure interface 7 by differently preconfiguring the cartridge 4 without reconfiguring the base structure 3.

As has also been explained, it may be the case that the standardized interface 6 is not fully utilized by every cartridge 4 for every processing step and differently utilized for differently processing steps.

According to one embodiment it is proposed, that the bioprocessing system 1 can move the standardized interface 6 with respect to the rest of the base structure 3 and/or the cartridges 4 or that the standardized interfaces 6 are immovable.

The bioprocess system 1 may comprise one or more input-output locations 46. These may be interfaces for the addition of consumables and/or initial cell cultures and/or the removal of packaged cell cultures during usage of the bioprocessing system 1.

In various embodiments the cartridges 4 have no energy source or only a battery that does not drive one of the functional devices 13. Additionally or alternatively the cartridges 4 may be partially or fully made out of plastic and/or 3D-printed and/or do not comprise any fluidic structure prior to being preconfigured.

It should be mentioned that the functional devices 13 are those devices that take part in the bioprocess not any elements with a function.

A cartridge 4 may have one wall covering its, in particular four, sides. It may have a floor and may have a cover. A cartridge 4 may be subjected to a defined atmosphere. A cartridge 4 may have a rectangular base form. A cartridge 4 that is not yet configured may have no functional elements. Electrical connections may be provided inside the wall and/or lid and/or floor of the cartridge 4.

Another teaching which is of equal importance relates to a cartridge 4 for use in the proposed method.

According to various embodiments it is proposed, that the cartridge 4 is preconfigured with a sterilized and sealed fluidic structure, in some embodiments, that the cartridge 4 is packaged as a ready-to-use component, in particular in a sterilized packaging.

Another teaching which is of equal importance relates to a base structure 3 for use in the proposed method.

Another teaching which is of equal importance relates to a bioprocessing system 1 for use in the proposed method.

1. A method for performing a bioprocess on liquid immune or naive cell cultures to obtain processed cell cultures, wherein the processed cell cultures are destined for autologous or allogenic cell therapy, wherein the bioprocess is performed on an integrated bioprocessing system, wherein the bioprocess comprises a sequence of processing steps, wherein the processing steps each comprise at least one operation,
wherein the bioprocess system comprises a base structure and preconfigurable cartridges, wherein the bioprocess system performs operations of the bioprocess via an interaction of the base structure with the cartridges,
wherein the cartridges and the base structure comprise matching standardized interfaces for an interaction of the base structure with the respective cartridge, wherein the bioprocess system performs at least two operations of the bioprocess inside at least two differently preconfigured cartridges by an interaction of the base structure with the cartridges via the same base structure interface and/or identical base structure interfaces and matching cartridge interfaces.

2. The method according to claim 1, wherein the two operations of the bioprocess are operations of different processing steps, or,
that the bioprocessing system performs at least two operations that are part of different bioprocesses in parallel in identically preconfigured cartridges, or,
that the bioprocessing system performs at least two operations that are part of different bioprocesses in parallel in differently preconfigured cartridges.

3. The method according to claim 1, wherein the bioprocessing system performs at least two bioprocesses or at least three bioprocesses or at least five bioprocesses or at least ten bioprocesses, each comprising at least two or at least three or at least four different processing steps in parallel, wherein the bioprocessing system performs at least one operation per processing steps, wherein the bioprocessing system performs those operations in at least two or at least three or at least four differently preconfigured cartridges per bioprocess.

4. The method according to claim 1, wherein the operations comprise operations of one or more of the processing steps “enrichment”, “selection”, “activation”, “loading”, “genetic modification”, “expansion”, “formulation”, “fill”, “wash” and “separation”, or,
that the processing steps comprise one or more of the processing steps “enrichment”, “selection”, “activation”, “loading”, “genetic modification”, “expansion”, “formulation”, “fill”, “wash” and “separation”.

5. The method according to claim 1, wherein the bioprocessing system comprises single step cartridges, whereby the bioprocessing system performs only operations of a single processing step inside the single step cartridges, or, that the bioprocessing system comprises shared cartridges, whereby the bioprocessing system performs operations of processing steps inside the shared cartridges.

6. The method according to claim 1, wherein the preconfigured cartridges comprise a preconfigured cartridge fluidic structure, or, that the preconfigured cartridges comprise a functional device for the operation performed inside the cartridge.

7. The method according to claim 1, wherein the standardized base structure interface comprises an active, energy transmitting interface, that the base structure via the active base structure interface and the matching cartridge interface transfers one or more of mechanical, pneumatic and electrical energy to the cartridge.

8. The method according to claim 1, wherein the bioprocessing system comprises receptacles, that the receptacles are preconfigured with receptacle fluidic structures for containing and providing or receiving the cell cultures or consumables that the bioprocessing system uses in at least some of the operations, and, that the receptacles comprise fluid interfaces identical to the fluid interfaces of the cartridges.

9. The method according to claim 1, wherein the bioprocessing system performs one or more or all operations of the processing step “enrichment”, the processing step and the processing step via the base structure inside a preconfigured filter cartridge with at least one filter as a functional device inside the cartridge.

10. The method according to claim 1, wherein the bioprocessing system performs one or more or all operations of the processing step “activation” via the base structure inside a preconfigured activation cartridge with at least one pump as a functional device inside or outside the cartridge.

11. The method according to claim 10, wherein the bioprocessing system performs one or more or all operations of the processing step “genetic modification” via the base structure inside a preconfigured genetic modification cartridge, and, that the activation cartridge and the genetic modification cartridge are one shared cartridge.

12. The method according to claim 9, wherein the bioprocessing system performs one or more or all operations of the processing step “genetic modification” via the base structure inside the filter cartridge as a shared cartridge.

13. The method according to claim 11, wherein the bioprocessing system performs the genetic modification by viral transduction or electroporation or nanoparticle-based delivery.

14. The method according to claim 1, wherein the cartridges or the receptacles are moved by the bioprocessing system automatically.

15. The method according to claim 1, wherein an electronic process control of the bioprocessing system may repeat a processing step or an operation if a criterion for the cell culture is not met during or after the processing step, thereby flexibly reconfiguring the bioprocess, or, that variations of processing steps may be performed via the same standardized base structure interface by differently preconfiguring the cartridge without reconfiguring the base structure.

16. The method according to claim 1, wherein the standardized interfaces are immovable.

17. The method according to claim 1, wherein the cartridges have no energy source or only a battery that does not drive one of the functional devices, or, that the cartridges are partially or fully made out of plastic or 3D-printed or do not comprise any fluidic structure prior to being preconfigured.

18. A cartridge for use in the method according to claim 1.

19. The cartridge according to claim 18, wherein the cartridge is preconfigured with a sterilized and sealed fluidic structure.

20. A bioprocessing system for use in the method according to claim 1.
